(19) United States
(12) Patent Application Publication
(10) Pub. No.: US 2026/0263112 A1
(43) Pub. Date: Sep. 10, 2026

(54) TROCAR/CANNULA ASSEMBLY

(71) Applicant: CERAMOX LLC, Syracuse, NY (US)

(72) Inventor: Michael T. Clarke, Manlius, NY (US)

(73) Assignee: CERAMOX LLC, Syracuse, NY (US)

(21) Appl. No.: 19/558,573

(22) Filed: Mar. 6, 2026

Related U.S. Application Data
(60) Provisional application No. 63/768,012, filed on Mar. 6, 2025.

(51) Int. Cl. A61B 17/34; A61B 17/00

(52) U.S. Cl. CPC A61B 17/3421; A61B 2017/00991

(57) ABSTRACT

A multi-part trocar assembly includes a distal tissue penetration section that has greater diameter than a central section, thereby providing a substantially smooth transition between a cannula and the trocar assembly to reduce friction of insertion and potential for damage to body tissues during insertion. In at least one embodiment, the trocar device is defined by a three (3)-component assembly in which the components fit in approximation to each other and when assembled provide an expanded, non-collapsible tip that contours smoothly and seamlessly with the cannula.

### CROSS REFERENCE TO RELATED APPLICATION

This application claims priority under 35 U.S.C. §119 and 35 U.S.C. § 120 to U.S. Patent Application Serial No. 63/768,012, filed on March 6, 2025, the foregoing application being incorporated by reference herein in its entirety.

### TECHNICAL FIELD

This application relates to a surgical tool and more specifically, a trocar device/assembly that permits easier and safer access to body tissues.

### BACKGROUND

Endoscopic surgery has revolutionized treatment of many medical conditions over at least the past fifty (50) years. As opposed to open surgery, endoscopic surgery is considerably less invasive, typically using only one or more small incisions to perform a medical procedure. This allows for a quicker recovery time for a patient with less pain and rehabilitation than that associated with open surgery.

Endoscopic surgery, however, still requires access to the interior of the body and as such necessitates the perforation of body tissues. Typically, the initial process of access to internal body structures involves insertion of a 'working portal.' The foregoing, commonly known as a cannula 10, FIG. 1, is typically a hollow tube of metal or plastic that may have specialized modifications depending on the surgical purpose. The hollow tube is further defined by an open distal end 14, which as shown is further defined by a peripheral taper. The cannula 10 is inserted into the body with the aid of a central rod known as a trocar 20, FIG. 2, or alternatively, a cannulated trocar 30, FIG. 3, each of which is either sharpened or at least shaped at its distal end 22 to enable perforation of the tissue with an applied force that is practical to enter the area of the body desired (cartilaginous joint, bursa, thorax, peritoneum, blood vessel are examples). An assembled cannula and trocar assembly 40 in accordance with the known art is shown in FIG. 4, including the prior art trocar 20. A cannulated trocar 30 can alternatively be substituted. Cannulated trocars 30 contain a small hole 32 at its distal most end that extends throughout the entire body of the trocar 30 such that the trocar 30 can be guided into position by a pre-positioned wire (e.g., a guide wire, not shown) placed with the aid of a cannulated needle (not shown) through which the guide wire passes. This process allows for reduced trauma by initially accessing the preferred area with a small needle that reduces the potential for tissue damage. More specifically and although not limiting, the guide wire (when used) is typically 0.5-2.0 mm in diameter, 100-300 mm in length, and made from a flexible metal alloy, such as Nitinol or stainless steel. The thin-walled, cannulated needle through which the guide wire is placed is typically 50-200 mm in length, and made from stainless steel, the needle having an internal diameter that is large enough to accommodate the guide wire. A typical endoscopic cannula placement workflow is as follows: (1) Placement of the tip of the cannulated needle into the area being operated on, typically aided by an x-ray fluoroscopic device for accurate placement, (2) placement and advancement of the guide wire through the cannulated needle to the body cavity being operated on, (3) removal of the cannulated needle over the guide wire, leaving the guide wire in place, (4) placement of the cannula/trocar assembly over the guide wire so that the tip enters the surgical area, (5) removal of the guide wire, and (6) removal of the trocar, leaving the cannula in its final position to enable use as the working portal.

With reference to FIG. 5, the typical profile of an assembled trocar 20 (cannulated trocar 30) and cannula 10 includes a transition zone or step/shoulder 44, which is formed at the distal end of the assembly 40. This transition zone can create difficulty in ease of tissue penetration of the subject, resulting in potential damage to bodily structures during tissue penetration, due to the presence of an abrupt discontinuity between the tapered distal end 14 of the cannula 10 and the outer surface of the trocar 20. The step 44 reduces the smoothness and ease of insertion of the cannula 10 and trocar assembly 40 and may allow uncontrollable thrusting of the unit into the body as the resistance of the body tissues suddenly gives out. Additionally, this step 44 can damage tissue even when insertion of the assembly 40 is otherwise controlled. For instance, in orthopedic surgery, such as during arthroscopy of the confined space of a cartilaginous joint, the formed step 44 provides a potential area of damage to the cartilage by direct abrasion. To this end, a trocar and cannula assembly that eliminates or greatly reduces the defined transition zone or shoulder/step 44 in presently known assemblies is greatly desired.

The problem in attempting to smooth or otherwise eliminate the step/shoulder between the trocar and the cannula, as described above, in order to allow easier tissue penetration of bodily tissues has been known to those of skill in the field for some time, and various attempts have been made to solve this problem. However, prior inventions or schemes have not yet adequately addressed the problem nor reduced the overall potential for soft tissue damage during trocar insertion for a variety of different surgical procedures.

For example, U.S. Patent Nos. 5,263,937 and 5,824,002 both describe trocar devices with a defined profile to reduce insertion force. These latter devices, however, are intended solely for laparoscopic procedures (penetration of the abdominal cavity) and further, each device requires a specialized trocar, as well as a specialized cannula. More specifically, the distal end of the cannula of these devices includes mobile members at its distal end that are pushed outwards radially during extraction of the trocar. This latter design is undesirable for at least three (3) reasons: (1) A custom cannula is required, meaning that contemporary or known cannulas already in use cannot be used; (2) the design requires a physical expansion of the diameter of the cannula in order to allow the trocar to be removed so that a tool or camera can be inserted. In many endoscopic procedures in which space is limited, such as in cartilaginous joints (arthroscopy of hip or ankle, for example), this expansion is undesirable as damage to the cartilage could occur during the radial expansion of the slotted members. In especially tight joints, in fact, the trocar may not be able to be removed as the members may not be able to be radially expanded; and (3) the mobile members of such a device as described by the above-noted patents must, by necessity, be more subject to failure by breakage than the non-expandable portion of the cannula. This latter reason is a concern for endoscopy in bodily areas in which hard or semi-hard tissue may be encountered, such as in arthroscopic surgery that is performed near bone and cartilage. Breakage of the trocar device may occur by impact or fatigue.

A number of other inventions have attempted to address the safety and ease of trocar and cannula insertion, using various other methods.

For example, U.S. Patent No. 5,350,393 describes a safety trocar penetrating instrument that describes a trocar cannula unit having a retractable sharp penetrating end. This instrument, however, does not appear to address the ease of tissue penetration by smoothing the trocar-cannula shoulder region. In addition, this specific instrument does not appear to be compatible with existing cannulas.

U.S. Patent No. 5,372,588 describes a trocar having a blunt tip in which the device has retractable cutting blades that expand during insertion, but retract when no insertion force is applied. Perhaps useful during penetration of large body cavities of soft tissue only, this apparatus not only requires a special cannula, but the apparatus also appears to be unusable in tight spaces where unwanted damage to tissue could occur, such as during arthroscopic joint surgery, in which damage to cartilage by the blades of this device could be a significant concern.

U.S. Patent No. 5,575,804 describes a safety penetrating instrument with a cannula. As described therein, the blade of the instrument that penetrates the tissue is withdrawn into the cannula after penetration of the abdominal cavity. The instrument described by this patent is defined by a complex construction and provides for a retractable penetrating trocar. Moreover, the apparatus described by this patent shares the same limitations as U.S. Patent No. 5,350,393, as previously discussed above.

U.S. Patent No. 6,436,119 B1 describes an adjustable surgical dilator. The device described by this latter patent is intended for spinal disc surgery in which a forcible expansion of the joint is required. This intention is in direct contrast to the aims of the herein disclosed apparatus in which no forcible expansion is desirable. The described dilator includes a central trocar and surrounding cannula. The cannula is again specialized and is described with having up to four (4) expandable members. In contrast to the novel device(s) described in this application, the expandable members of the specialized cannula are not seamlessly opposed to each other and are separated by gaps. These gaps are undesirable for endoscopic surgery in many body tissues as they can abrade, cut or otherwise damage sensitive tissue during movement while performing necessary surgical procedures.

### BRIEF DESCRIPTION

Accordingly and to address the above-noted needs in the field, a trocar device/assembly is provided that provides improved insertion force for insertion into the body. According to at least one embodiment, the trocar device/assembly can be constructed of at least three (3) inter-related members or components. When retracted, these components are able to be engaged within an outer or exterior cannula. For purposes described herein, the cannula can be conventional in design. Alternatively, the cannula can be specially or specifically designed. When the components of the trocar device/assembly are advanced axially into an active predetermined or deployed position relative to a patient during a surgical procedure, the distal end of the trocar device provides for an expanded tip that provides a smooth transition from trocar to cannula and substantially eliminates the abrupt and discontinuous shoulder or step commonly formed in existing trocar assemblies. As a result, there is a significantly decreased potential for damage of internal tissues when the herein described trocar device/assembly is inserted for penetration.

In accordance with at least one embodiment, the members/components of the herein described trocar device/assembly are formed such that an outermost component or member is positioned first, followed by a central member, and finally an innermost member of the assembly. To coordinate the components in order to create an expanded trocar tip and also to avoid potential jamming of the trocar assembly, the insertional arrangement of the herein described trocar device/assembly may be facilitated using a differential spring mechanism according to at least one version, or alternatively using another suitable system. Once the inter-related members of the trocar device/assembly are suitably positioned axially and rotationally, the components can then be locked together by a suitable mechanism, which can be provided, according to at least one embodiment at a proximal end of the assembly.

Once suitably positioned and following the removal of a previously placed guide wire, if used, the trocar assembly can be removed from the patient, leaving only the outer cannula in place. The expanded trocar tip is non-compressible and during both insertion and removal does not substantially expand, nor does the trocar device require expansion of the outer cannula for purposes of removal. In one or more embodiments, one or more of the components of the trocar assembly can be formed in a "flexed" condition, in order to avoid potential issues when removing the trocar assembly from the cannula.

According to at least one version, the members of the trocar device/assembly can be concentrically aligned with one another and independently movable until locked in place. Alternatively, one or more members/components of the trocar device/assembly can be aligned in a parallel or concentric arrangement in order to create a suitably expanded trocar tip having a substantially smooth transition with the outer cannula.

Proximally, the components of the trocar device/assembly can be formed with interlocking features, according to at least one embodiment, such that the components of the device/assembly can connect and lock to one another, as well as to the cannula in order to provide ease of insertion. The proximal ends of each of the components can be manufactured with protruding and hollowed regions that allow and limit both linear and rotary motion relative to each to a required degree.

As noted, a multi-part trocar device/assembly according to aspects of this disclosure is configured and sized to be movable within an outer cannula, the assembly being designed to penetrate inner cavities or tissues of the body. Prior to insertion into the body of a subject and according to at least one version, the trocar device/assembly is axially advanced within the hollow interior of the cannula and comprises at least three (3) members, which when assembled, combine to create an expanded distal section (a blade tip) that is at least larger in diameter than the circumference of a middle section of the trocar assembly, and preferably at least as wide in diameter as the external diameter of the outer cannula into which the trocar assembly is introduced. This expanded distal section creates a reduced shoulder (i.e., a smooth transition zone) between the trocar assembly and cannula, thereby improving ease of insertion and reducing potential damage to bodily tissues. According to one or more embodiments, the distal sections of the first two members or components of the trocar device/assembly are configured to have a greater external diameter than their corresponding middle or intermediate sections, and typically an external diameter that essentially matches that of the outer cannula.

Despite their larger outer diameter, the distal ends of the first two components of the herein described trocar device/assembly are sufficiently flexible to permit radial compression, thereby enabling passage of the first and second components through the interior of the outer cannula. As each of the distal ends of the first and second components are axially advanced through the distal end opening of the hollow cannula, these members can each expand and are intimately associated with each other and the outer cannula to create a locking arrangement. In at least one version, the outer cannula is defined by a tapered distal end, wherein the distal sections of at least one of the components of the trocar device are defined by a tapered proximal surface. The third member or component of the trocar device/assembly can then be passed through the end of the cannula, this latter component being non-compressible and preventing collapse of the first two components to which the third component also becomes intimately associated. When deployed, the trocar device/assembly is temporarily adherent to the outer cannula at its proximal end during insertion into the body tissues, but the trocar device/assembly can be subsequently removed, leaving only the outer cannula in place. The remaining hollow cannula creates a working portal to the inside of the body through which a diagnostic or therapeutic surgical procedure can be performed. For example, the cannula can typically be used in order to house a camera or allow the passage of various tools or surgical implants that are designed for endoscopic use.

According to at least one embodiment, the proximal section of the trocar device/assembly is sized and configured to allow orientation and temporary fixation of the trocar device/assembly. In at least one version, for example, the components of the trocar assembly are configured for rotation within the cannula, as well as axial advancement. In at least one version, a locking mechanism can further be utilized to enable and guarantee the temporary fixation. According to at least one embodiment, the proximal end of the herein described trocar device/assembly can include an integrated mechanism having springs with differential compressive moduli that allows the individual components of the trocar device/assembly to be advanced and positioned in a predetermined order so that the trocar tip can be expanded beyond the distal end of the outer cannula. Otherwise, there is a potential that the various trocar components may jam together within the outer cannula if not advanced or retracted in a coordinated manner. The spring-loaded mechanism with different compressive moduli or other similar mechanism(s) can further allow for disassembly of the trocar device/assembly from the body in a similar, but reverse manner.

These and other features and advantages will be readily apparent from the following Detailed Description, which should be read in conjunction with the accompanying drawings.

### BRIEF DESCRIPTION OF THE DRAWINGS

The drawings show embodiments of the disclosed subject matter for the purpose of illustrating the invention. However, it should be understood that the present application is not limited to the precise arrangements and instrumentalities shown in the drawings, wherein:

FIG. 1 is a perspective view of a prior art cannula;

FIG. 2 is a perspective view of a prior art trocar;

FIG. 3 is a perspective view of another prior art trocar being configured for access by a guide-wire (not shown) through a cannulated distal tip opening;

FIG. 4 is a perspective view of the prior art trocar of FIG. 2 housed within the prior art cannula of FIG. 1;

FIG. 5 is an enlarged partial side elevational view of the distal end of the prior trocar/cannula assembly of FIG. 4, including a shoulder/step in a transition zone between the trocar and outer cannula;

FIG. 6 is a front perspective view of a trocar assembly in accordance with aspects of this disclosure, the trocar assembly being mated within the prior art cannula of FIG. 1;

FIG. 7 is an enlarged side perspective view of the distal end of the trocar assembly and cannula of FIG. 6, as mated within the prior art cannula;

FIG. 8 is an enlarged top view of the distal end of the trocar assembly and cannula of FIGS. 6 and 7;

FIG. 9 is an enlarged side elevational view of the distal end of the trocar assembly and cannula; the view being taken 90 degrees relative to FIG. 8;

FIG. 10 is a front perspective view of a first component of the trocar assembly of FIGS. 6-9 made in accordance with an embodiment;

FIG. 11 is a side elevational view of the first component of FIG. 10;

FIG. 12 is an enlarged perspective view at the distal end of the first component of FIGS. 10 and 11;

FIG. 13(a) is an enlarged side elevational view of the first component of FIGS. 10- 12;

FIG. 13(b) is an enlarged side elevational view of the first component of FIGS. 10- 13(a), but with the distal section being manufactured in a flexed condition in accordance with an alternative embodiment;

FIGS. 13(c) and 13(d) are enlarged front perspective views of a first component in accordance with one or more embodiments of this disclosure;

FIG. 14(a) is a front perspective view of a second component of the trocar assembly of FIGS. 6-9, made in accordance with an embodiment;

FIG. 14(b) is an enlarged front perspective view of the proximal section of the second component of FIG. 14(a);

FIG. 15(a) is a side elevational view of the second component of FIGS. 14(a) and 14(b);

FIG. 15(b) is an enlarged side elevational view, taken in section, of the proximal section of the second component of FIG. 15(a);

FIG. 16 is an enlarged perspective view of the distal end of the second component of FIGS. 14 and 15;

FIG. 17(a) is an enlarged side elevational view of the second component of FIGS. 14-16;

FIG. 17(b) is an enlarged side elevational view of the second component but with the distal section being manufactured in a flexed condition, in accordance with an alternative embodiment;

FIG. 18(a) is a front perspective view of a third component of the trocar assembly of FIGS. 6-9, made in accordance with an embodiment;

FIG. 18(b) is an enlarged front perspective view of the proximal section of the third component of FIG. 18(a);

FIG. 19(a) is a side elevational view of the third component of FIGS. 18(a) and 18(b);

FIG. 19(b) is an enlarged side elevational view, taken in section, of the proximal section of the third component of FIG. 19(a);

FIG. 19(c) is a perspective view of a locking pin used in conjunction with the third component and in accordance with an embodiment;

FIG. 19(d) is a side elevational view of the locking pin of FIG. 19(c);

FIG. 20 is an enlarged perspective view of the distal end of the third component of FIGS. 18 and 19;

FIG. 21 shows an enlarged side view of the distal end of the third component of FIGS. 18-20;

FIG. 22 shows an enlarged perspective view of the first component of FIGS. 10- 13(a) in an active position relative to the prior art cannula of FIG. 1;

FIG. 23 is a perspective view of the first and second components of FIGS. 10-17(a) in an active position relative to the prior art cannula of FIG. 1;

FIG. 24 shows an enlarged perspective view of first, second and third components of FIGS. 10-21 in an active position relative to the prior art cannula of FIG. 1;

FIG. 25 is a perspective view of a fully expanded trocar assembly in accordance with an embodiment, this assembly being depicted without a cannula;

FIG. 26 is a perspective view of the trocar assembly of FIG. 25 in a partially compressed condition; the assembly being depicted without a cannula;

FIG. 27(a) is a perspective view of the trocar assembly of FIGS. 25 and 26 in a fully compressed condition, the assembly also being depicted without a cannula;

FIGS. 27(b)-27(d) are sequential partial side elevational views of a locking mechanism for the trocar assembly in accordance with an embodiment;

FIG. 28(a) is a vertical longitudinal sectioned view of a distal section of the trocar assembly of FIGS. 25-27, relative to an outer cannula;

FIG. 28(b) is a horizontal sectioned view of a distal section of the trocar assembly of FIGS. 25-27, relative to an outer cannula;

FIG. 29 is a cross section view taken at an intermediate or middle portion of the trocar assembly of FIGS. 25-28(b), and within the outer cannula;

FIG. 30 is a perspective view of a first component of a trocar assembly made in accordance with another embodiment.

FIG. 31 is an enlarged perspective view of the distal section of the first component of the trocar assembly of FIG. 30;

FIG. 32 is a perspective view of the second component of a trocar assembly in accordance with another embodiment;

FIG. 33 is an enlarged perspective view of the distal section of the second component of the trocar assembly of FIG. 32;

FIG. 34 is a perspective view of the trocar assembly of FIGS. 30-33, shown in a fully expanded condition, without a cannula;

FIG. 35 is a perspective view of the trocar assembly of FIGS. 30-34, shown in a partially compressed condition, without a cannula;

FIG. 36 is a perspective view of the trocar assembly of FIGS. 30-34, shown in a fully compressed condition, without a cannula;

FIG. 37 is a cross-section view taken through the middle sections of the trocar assembly of FIGS. 30-36, as shown with a cannula;

FIG. 38 is a side view of a longitudinal sectioned view of a trocar assembly in accordance with an embodiment, the assembly being fully expanded relative to an outer cannula as coordinated by a spring-loaded mechanism.

FIG. 39 is a side view of a longitudinal section view of the trocar assembly of FIG. 38 fully compressed within the outer cannula using the spring-loaded mechanism;

FIG. 40 is a side perspective view, partially in section, of the trocar assembly of FIG. 38, fully expanded within the outer cannula;

FIG. 41 is a side perspective view, partially in section, of the trocar assembly of FIG. 39, fully compressed within the outer cannula, and in a locked position

FIG. 42 is a side perspective view of an embodiment of a portion of a cannula showing a seat region;

FIG. 43 is a side perspective view of the embodiment of the portion of the cannula from FIG. 42 with an embodiment of a sheath positioned on the seat region; and

FIG. 44 is a side perspective view of the embodiment of the sheath from FIG. 43.

### DETAILED DESCRIPTION

The following describes one or more embodiments of a surgical tool apparatus, and more specifically a trocar/cannula assembly adapted or configured for use in various surgical procedures. A primary goal or aim of the herein described tool apparatus and related methods is to prevent or minimize damage to body tissues of a patient while making a suitable incision by the creation of a trocar blade tip that is caused to radially expand when advanced axially from the distal end of a hollow cannula. This expansion creates a transition zone between the trocar blade tip and the cannula that is substantially smooth and lacking in abruptness.

It will be readily apparent that there are a number of modifications and variations of the herein described surgical tool that can be employed, depending upon, for example, a specific surgical procedure. Over the course of the following description, a number of terms are used to provide an adequate frame of reference for the accompanying drawings. These terms, which include "first," "second," "third," "distal," "proximal," "middle," "intermediate," and the like, are not intended to limit the overall scope of the herein described apparatus or related methods, except where so specifically and positively indicated. Moreover, the accompanying drawings are provided to illustrate salient features of the herein described surgical apparatus and related methods of use, but should not be relied upon by the reader for scaling purposes.

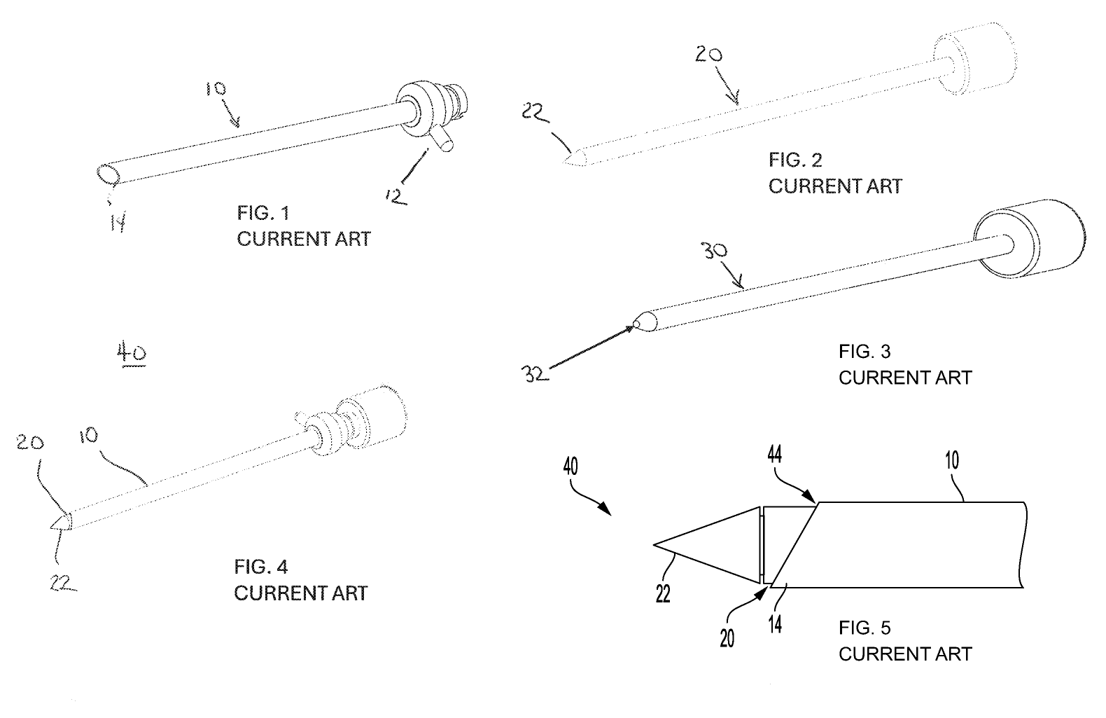

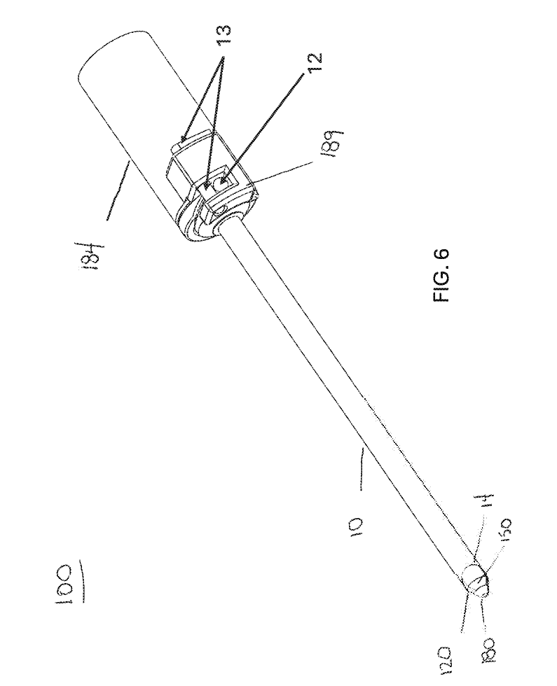

With reference to FIGS. 6-9, a first embodiment of a trocar/cannula assembly 100 is depicted that substantially reduces or eliminates the step or shoulder traditionally found at the transition zone between the trocar blade tip and outer cannula in known or conventional assemblies to date. In brief, the herein described trocar device/assembly 100 is defined, according to this embodiment, by three (3) inter-related components, herein referred to as the "first component" 120, the "second component" 150 and the "third component" 180, respectively. In brief, the herein described trocar 100 (also referred to herein as a "trocar assembly" or "trocar device") is designed to fit within currently available hollow cannulas, such as cannula 10, FIG. 1, without any significant modification. FIG. 6, depicts a "deployed" position of the trocar assembly 100 with the distal portions of the trocar assembly 100 being axially advanced beyond the tapered distal end 14 of the cannula 10 and in which the cannula 10 has been locked in place.

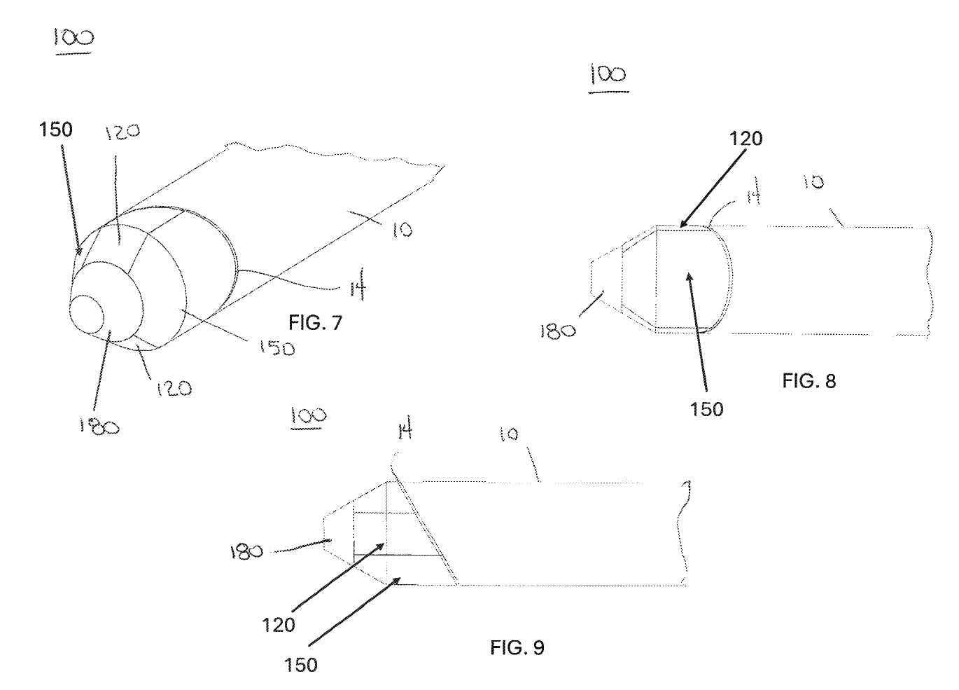

FIGS. 7-9 show views of the tip of the trocar assembly 100 and cannula 10 with the trocar assembly extending or advanced beyond the distal end 14 of the cannula 10 and in the deployed position. As discussed in greater detail and unlike conventional trocar assemblies, there is no discernable step formed between the trocar blade tip and a conventional outer cannula, the latter having the tapered distal end 14. Alternatively, the three-part collapsible unit/assembly 100 in accordance with this embodiment may be formed to conform with a specialized cannula (not shown) that is specifically designed for the surgical purpose.

As indicated, the trocar assembly 100 is defined by multiple components, herein also referred to as "parts" or "members" for purposes of this discussion. As described herein, these components are sized and configured to slide and/or rotate within each other in a nested configuration in order to create an expanded trocar tip for deployment to an active (deployed) position beyond the distal opening or end 14 of the hollow cannula 10. Each of the components in accordance with this embodiment is now described in greater detail.

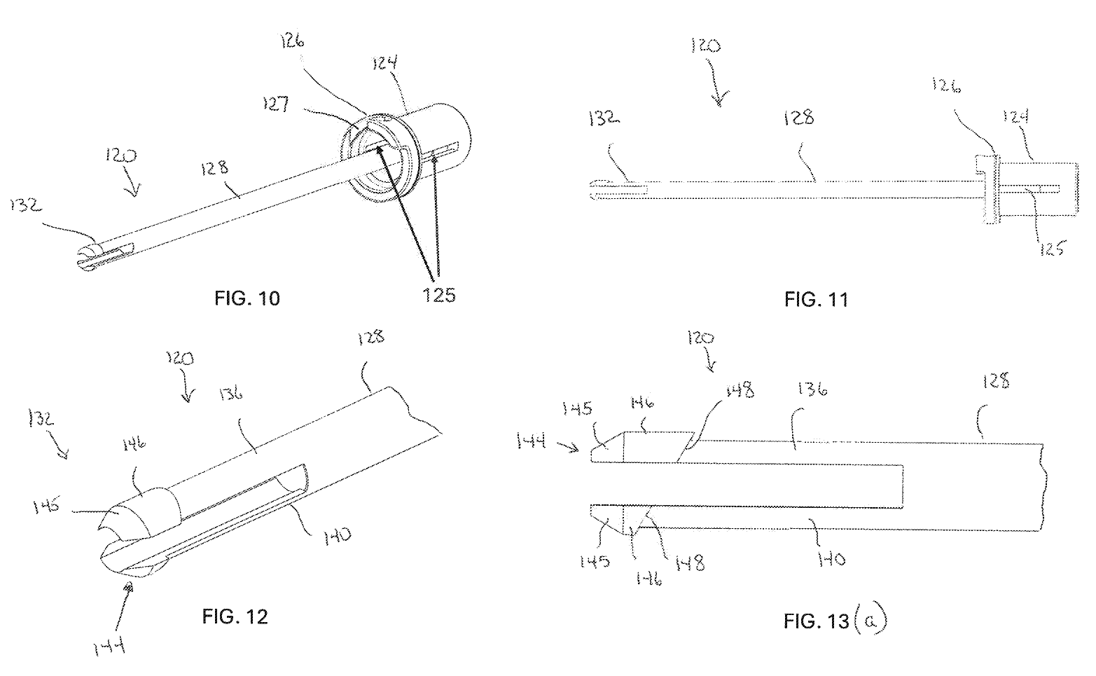

FIGS. 10 - 13(a) depict the first component 120 of the trocar or trocar assembly 100 in accordance with this embodiment. More specifically, the first component 120 is defined by a substantially elongate member having respective proximal, middle and distal sections 124, 128, 132 along its length that is further defined by open distal and proximal ends and a hollow interior. The first component 120 may be manufactured from a suitable biocompatible material, such as stainless steel or plastic. The first component 120 may be manufactured as a single unitary member, but alternatively may also be manufactured in multiple parts and assembled using mechanical or adhesive methods of connection using multiple materials and adhesives. The latter simplifies its manufacture. The wall thickness of the first component 120, most particularly the long middle section 128, must be of a sufficient dimension to withstand the stress of insertion and use. This thickness may range from 0.1 mm to 5 mm or more, for example, depending on the cannula size being used. Cannula diameters can range from 2-15 mm for example, depending on the area of the body being accessed. Large cavities, such as the peritoneal cavity, would allow use of a large cannula, whereas tighter spaces such as the knee joint are frequently accessed with cannulas that are 6.5-9 mm in diameter. The hip joint is a more difficult joint to enter than the knee joint, with limited space available in which cannulas in the range 4.5-6.5 mm are typically used. The ankle is a joint with even more limited space than any of the foregoing, in which a 2.7 mm cannula is frequently used.

The proximal section 124 of the first component 120 is configured so that the proximal section 124 can contour with the appropriate shape of a corresponding cannula 10, both distally and proximally. More specifically, the proximal section 124 is defined by a substantially cylindrical configuration that is essentially hollow. A raised distal end 126 includes a bayonet slot 127 disposed about the circumference of the raised distal end 126. The foregoing feature allows the first component 120 to align longitudinally and radially to a protrusion 12, FIG. 1, of the current art cannula 10, FIG. 1, which radially extends from a proximal end thereof. Protrusion 12 is seated fully when at the blind end of the bayonet slot 127. As discussed in greater detail below, this positioning is necessary to allow the distal tip and more specifically portion 146 to align properly with the beveled end 14 of the current art cannula 10, such that the trocar tip can expand properly and that a smooth transition is also created. The remainder of the proximal section 124, according to this specific embodiment, is defined by a continuous cylindrical wall with the exception of a pair of axial slots or channels 125, which are formed on opposite ends of the proximal section 124, each of the channels 125 extending over substantially the length of the proximal section 124. At least one axial slot 125 is necessary, but multiple slots along the circumference of the component 120 may be present. The raised distal end 126 of the proximal section 124 is defined by an expanded outer diameter relative to the remainder of the substantially cylindrical proximal section 124.

The middle or intermediate section 128 of the first component 120 according to this embodiment is defined by a cylindrical tubular configuration bridging the proximal and distal sections 124, 132, to allow passage through the hollow interior of the cannula 10, and further to allow passage of the second component 150 within the interior of the first component 120. More specifically, a proximal end of the tubular intermediate section 128 extends into the interior of the proximal section 124, as most clearly shown in FIG. 10.

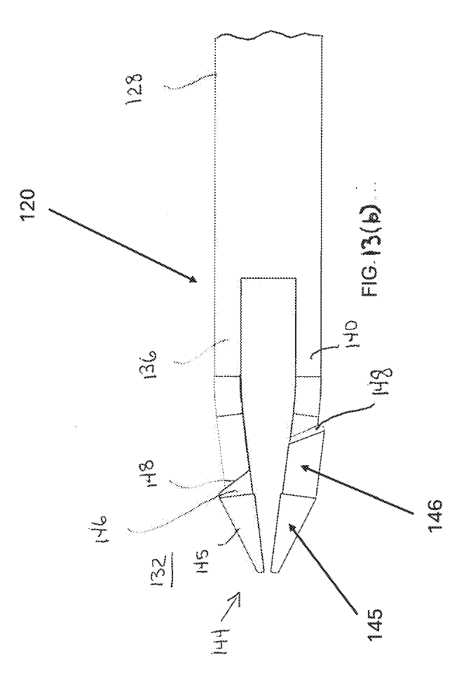

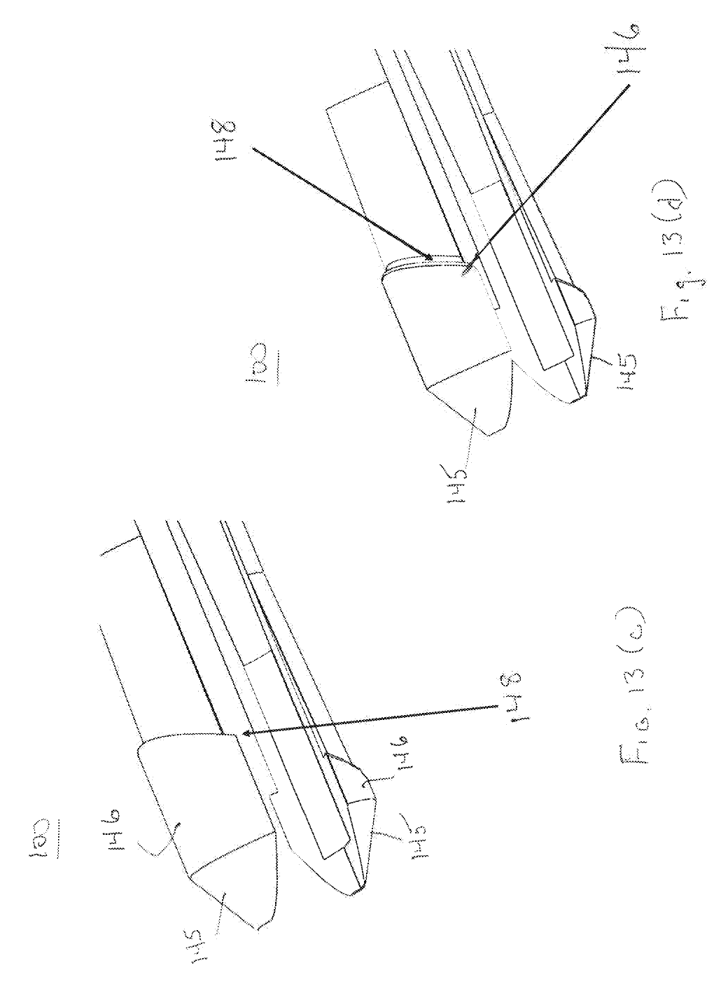

The distal section 132 of the first component 120 is also defined as a hollow tubular extension extending distally from the middle section 128, in which annular wall portions of the tubular configuration are removed starting at the distalmost end of the distal section 132. This removal results in a pair of gaps or slots that axially extend to the distal end of the intermediate or middle section 128. The removal of these annular wall portions creates a parallel set of flexure- like sections 136, 140, which are in spaced and parallel relation to one another, as shown in FIGS. 12 and 13(a). The distal most end of each of the flexure-like members 136, 140 is further defined by a blade portion 144 made up of a frusto-conical distal tip 145 extending proximally to a raised cylindrical portion 146. According to this embodiment, the raised cylindrical portions 146 of each of the flexure-like members 136, 140 are commonly defined by a tapered proximal end surface 148, wherein the overall length of the cylindrical portion 146 of one of flexure-like members 136 is axially longer than the overall length of the remaining flexure-like member 140. The tapered proximal end surface 148 creates an angled annular protrusion extending above the outer surface of the remaining proximal portion of the distal section 132 of the first component 120, which is defined by a cylindrical cross section and having an outer diameter, the latter of which is consonant with that of the middle section 128, as best shown in FIGS. 12 and 13(a).

Alternatively, the distal section can be manufactured, as shown in FIG. 13(b), in which the tip portions 145, 146 are manufactured in an initially flexed condition.. For purposes of this alternative embodiment, similar components or features are labeled with the same reference numerals for purposes of clarity. This latter embodiment may be advantageous in that the expanded tip may be less likely to catch and "hang up" on the beveled tip 14 of the cannula 10 during removal of the trocar assembly 100 as the first component 120 elastically returns to its starting (flexed) position during disassembly. In accordance, with yet another embodiment and with reference to FIGS. 13(c) and 13(d), at least a small portion of the proximal end surface 148 can be formed, as shown, with a cut out and filets to prevent potential impingement against the beveled distal end of the cannula 10, and further prevent resistance when the first component 120 is rotated for purposes of removal from the cannula 10.

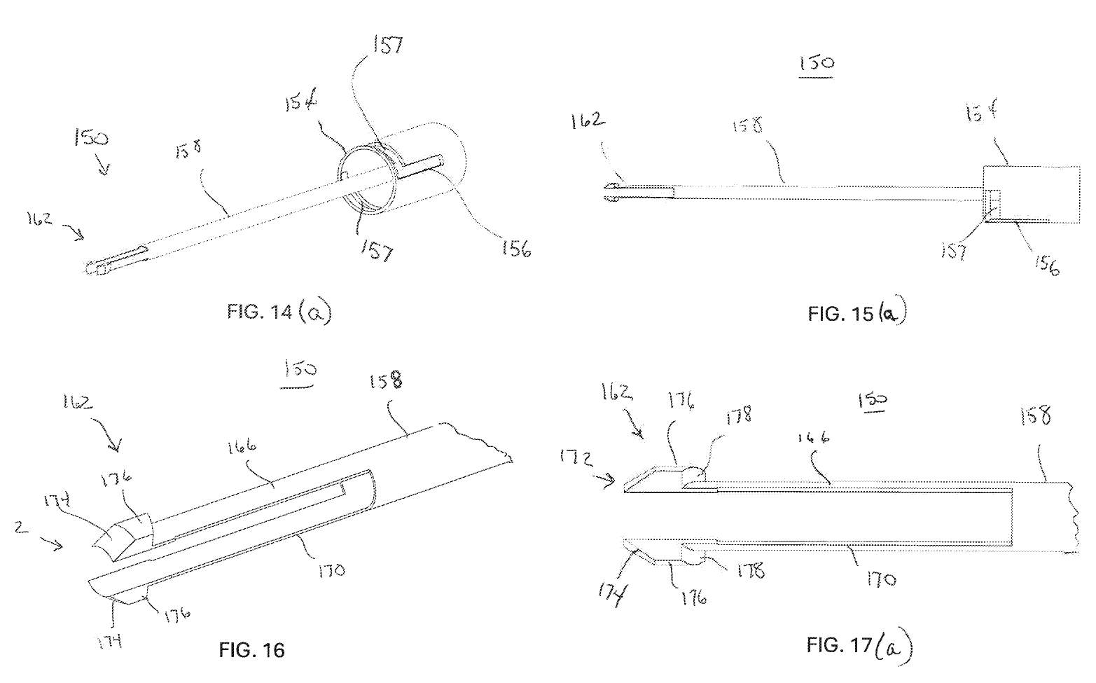

FIGS. 14(a)-17(a) illustrate the second part or component 150 of the trocar assembly 100 in accordance with this embodiment. More specifically and like the first component 120, the second component 150 is also an elongate member having a hollow interior, as well as proximal, middle and distal sections 154, 158 and 162, respectively. The second component 150 may be manufactured from a suitable biocompatible material, such as stainless steel or plastic, and may or may not be made from the same material as the first component 120. The second component 150 may also be manufactured as a single member, or alternatively in multiple parts and assembled having mechanical or adhesive methods of connection and using multiple materials and adhesives. The latter simplifies its manufacture. Similar to that of the first component 120, the wall thickness of the second component 150, most particularly the long middle section 158, must be of a sufficient dimension to withstand the stress of insertion and use. This thickness may range from 0.1 mm to 5 mm, for example, depending on the cannula size being used.

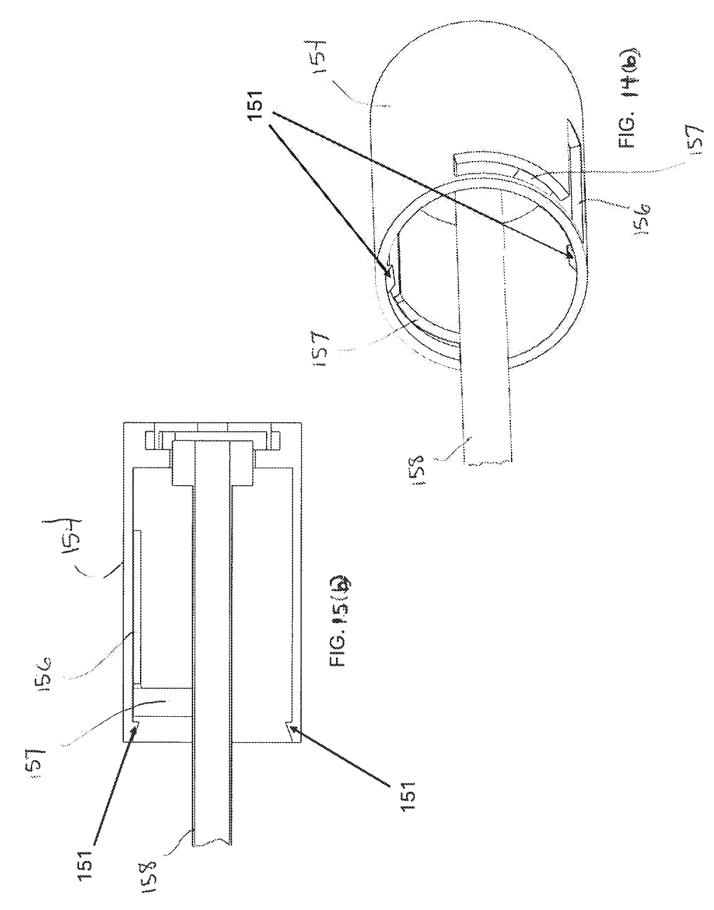

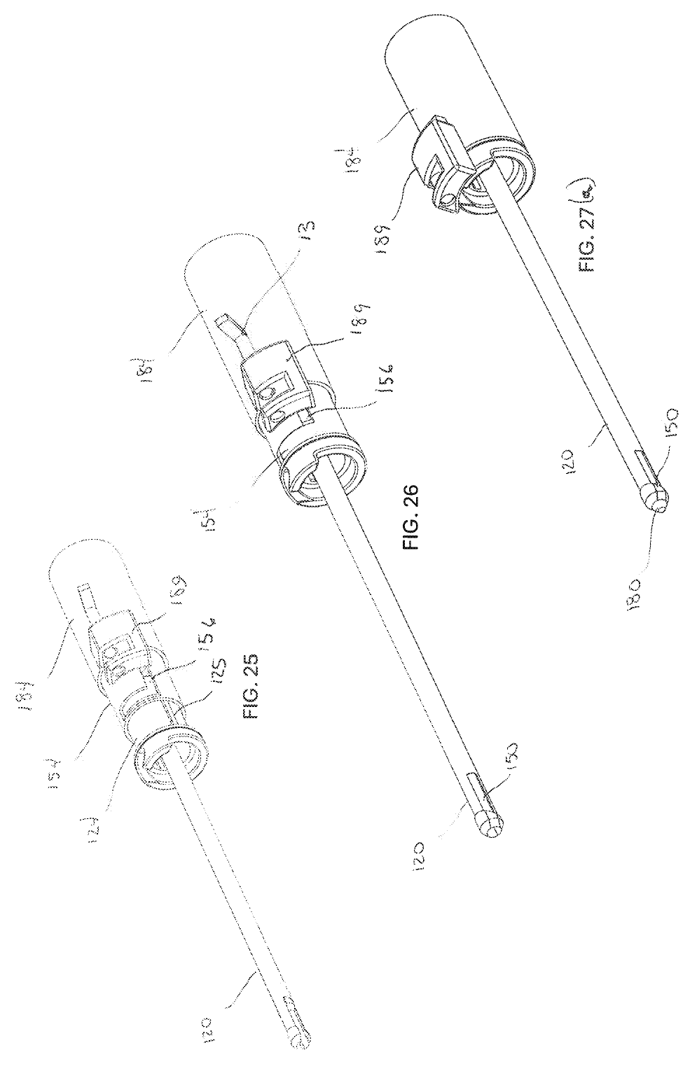

The proximal section 154 is designed and appropriately sized to slide over the outer surface of the proximal section 124 of the first component 120, as shown most clearly in FIG. 25. More specifically and according to this embodiment, the proximal section 154 is defined by a substantially cylindrical and hollow configuration. As shown more specifically in FIGS. 14(b) and 15(b), a pair of outwardly tapered protrusions 151 are formed on the inner diameter of the hollowed section in which the protrusions 151 are diametrically spaced in relation to one another, according to this embodiment. The protrusions 151 are sized and configured such that they engage the axial channels 125 formed in the proximal section 124 of the first component 120 and provide limits on rotational as well as linear (axial) movement. The tapering cross-sectional or wedge-like shape of the protrusions 151, according to this specific embodiment, allows the protrusions 151 to snap into place over the first component 120 by temporary deformation of the hollowed proximal section 124 during assembly, but also prevents easy or unintended disassembly of the first and second components 120, 150.

A pair of annular slots or channels 157 are formed at the distal end of the proximal section 154, as well as a pair of linear slots or channels 156 that are orthogonally formed at one end of one of the annular slots 157 and forming an L-shaped configuration. According to this embodiment, the axial channels 156 extend proximally along the major axis of the proximal section 154.

The middle or intermediate section 158 of the second component 150 is defined by a substantially tubular cross section, which bridges the proximal and distal sections 154, 162. More specifically, the tubular intermediate section 158 proximally extends into the hollow cylindrical proximal section 154, the latter having a significantly greater diameter than the remainder of the second component 150. The intermediate portion 158 is sized and configured to allow passage through the hollow interior or core of the first component 120, and further to allow passage of the third component 180 within the second component 150, as described in greater detail below.

The distal section 162 of the second component 150 according to this embodiment is an axial extension of the tubular cross section of the middle or intermediate sections 158 with the exception of removed annular wall portions, thereby defining a pair of flexure-like members 166, 170, which are in spaced and parallel relation to one another. Each of the flexure-like members 166, 170 are defined by a blade head 172 formed at the distal most end of the second component 150. The blade head 172 includes a beveled distal tip 174 at an open end of the second component 150 that extends to a raised annular portion 176. The raised annular portion 176 includes a proximal end surface 178 downwardly extending to the cylindrical outer surface of the second component 150. The angle of engagement of the proximal end surface 178 is pictured at a right angle to the shaft, but may be tapered, if required. Otherwise, the flexure-like members 166, 170 of the second component 150 are sized to "fit" within the gaps or openings formed in the distal section 132 of the first component 120, thereby forming a fully cylindrical distal section of the assembly 100, when assembled and as described in greater detail below.

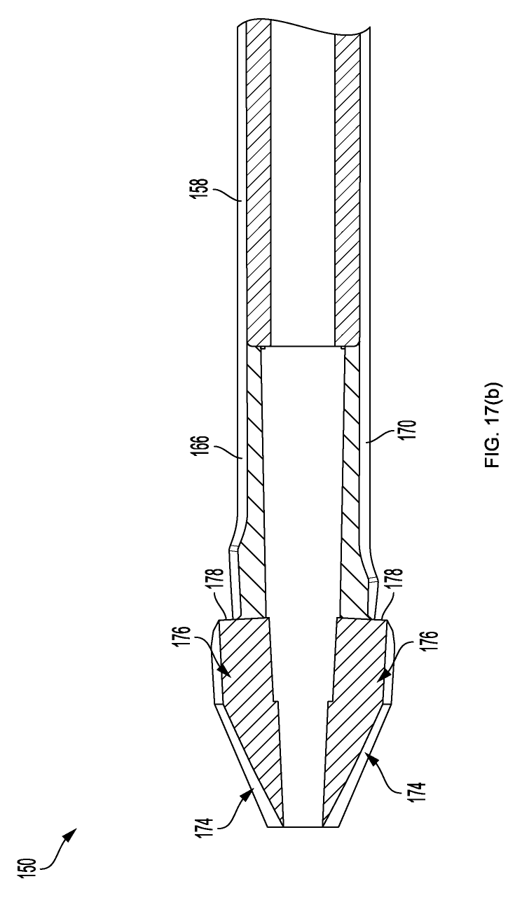

Alternatively, the distal section 162 can be manufactured as shown in FIG. 17(b), in which the distal section of the second component 150 is manufactured in a flexed condition. With regard to this latter embodiment and as shown in FIG. 17(b), similar parts or features are herein labeled with the same reference numbers for the sake of clarity. This latter configuration may be advantageous in that the expanded distal end may be less likely to catch and hang up on the beveled distal tip 14 of the cannula 10 during removal of the trocar assembly 100, as the component tip springs back to its original position away from the cannula edges.

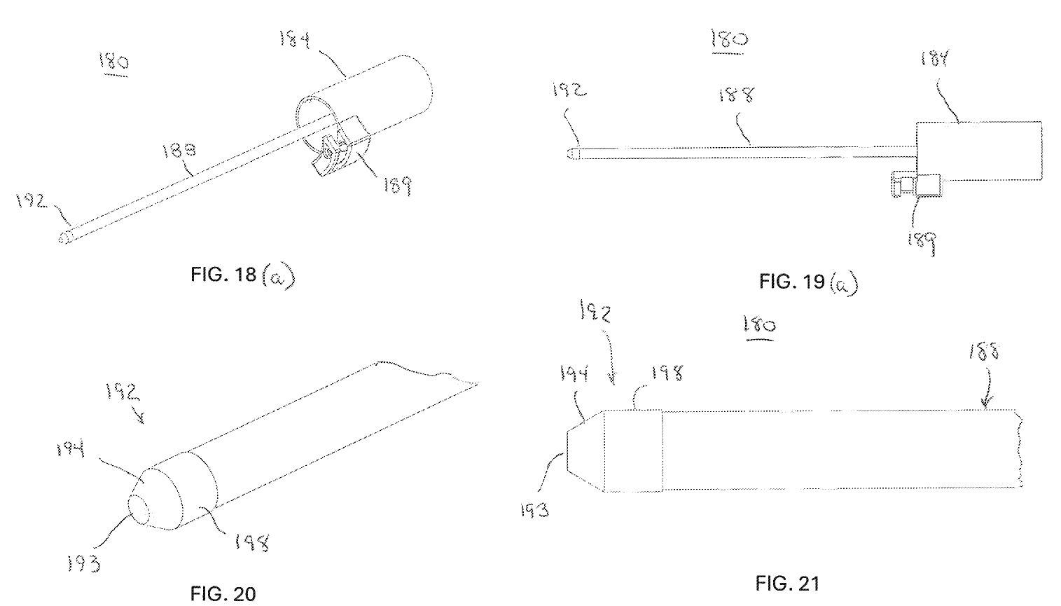

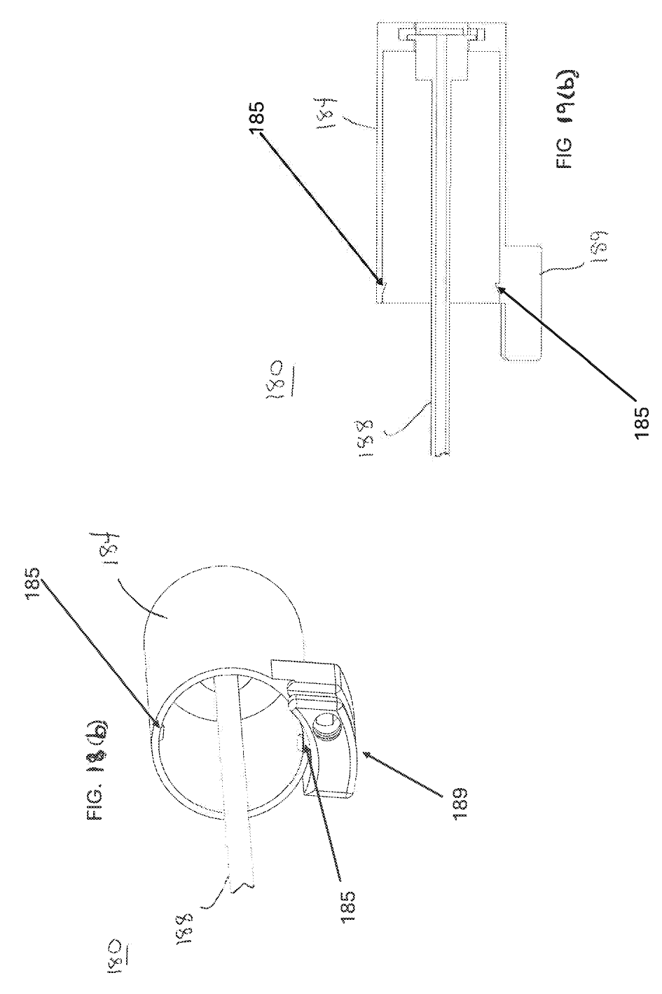

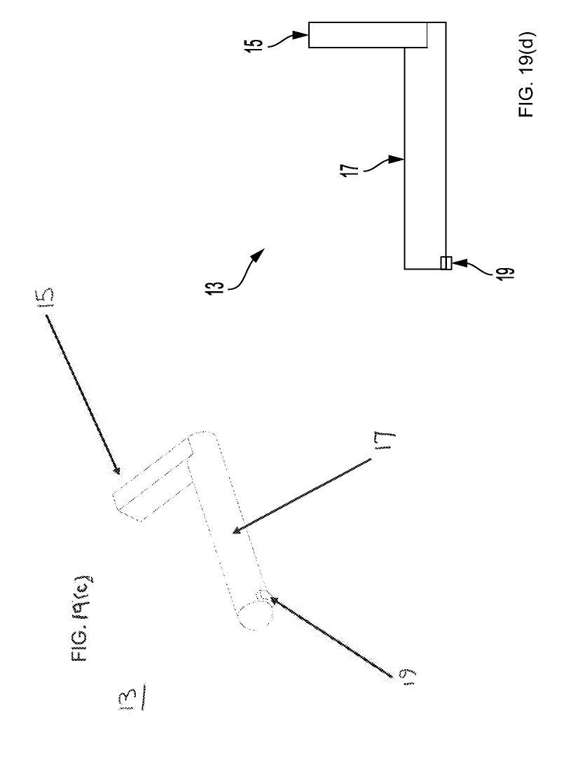

FIGS. 18(a)-21, depict the third component 180 of the trocar assembly 100 in accordance with this specific embodiment. The third component 180 is defined by a substantially elongate member that includes respective proximal, middle and distal sections 184, 188, and 192. The proximal section 184 is designed to slide over the exterior surface of the proximal section 154 of the second component 150, as shown more clearly in FIGS. 25 and 26. More specifically, the proximal section 184 is defined by a substantially cylindrical configuration that is essentially hollow. The proximal section 184 further contains features that allow rotational and linear orientation to the proximal section 124 of the first component 120. More specifically and according to this embodiment, with reference to FIGS. 18(b) and 19(b), a pair of inwardly tapered protrusions 185 are formed on the inner diameter of the hollowed proximal section 184 that are positioned in diametrical opposition to one another. These protrusions 185, which are defined by a wedge-like shape according to this embodiment, are such that they engage the channels or slots 156 and 157 formed on the proximal section 154 of the second component 150 and provide limits on rotational and linear (axial) movement therebetween. More specifically, the tapered cross-sectional shape of the wedge-shaped protrusions 185, according to this embodiment, allows the protrusions 185 to snap into place over the first component 120 by temporary deformation of the hollowed proximal section 124 during assembly, but also prevent easy disassembly of the first and second components 120, 150. As noted, the axial slots 156 are designed to engage the protrusions 185 of the third component 180. This latter engagement allows rotational alignment of the third component 180 with respect to the second component 150. When the second and third components 150, 180 are compressed together, the protrusions 185 of the third component 180, as shown in FIGS. 18(b) and 19(b), travel distal within each of the axial slots 156 of the second component 150 until reaching the annular slots 157 at which point, the third component 180 is able to be rotated into its final position, which is the engagement of the protrusion 12 of current art cannula 10.

Additionally, an expansion 189 is formed on the outer surface of the proximal section 184 at the distal end thereof. This expansion 189 includes a slotted or hollowed region 190 that is configured to receive the radially extending protrusion 12 of the trocar 10, FIG. 1, as well as the end of a locking pin 13, the latter being shown in detail in FIGS. 19(c) and 19(d). More specifically, the locking pin 13, according to this embodiment, is defined by a substantially L- shaped configuration, including a handle portion 15 and a pin portion 17 that orthogonally extends from one end of the handle portion 15. The pin portion 17 is sized to fit within an opening (not shown) formed in the expansion 189 that extends into the slotted or hollowed portion 190 of the expansion 189 in order to specifically restrict movement of the protrusion 12 after the third component 180 has been rotated or twisted into a locking position, and after the trocar assembly 100 has first been fully compressed and the trocar blade tip has been expanded.

The middle or intermediate section 188 of the third component 180 is hollow and substantially tubular in order to bridge the proximal and distal sections 184, 192 wherein the intermediate section 188 extends proximally within the hollow cylindrical proximal section 184, as best shown in FIGS. 18(a) and 18(b). The middle section 188 is further configured and sized to allow passage through the hollow core of the second component 150. The distal section 192, according to this embodiment and best shown in FIGS. 20 and 21, is defined by a frusto-conical distal tip 194 having a distal tip opening 193, in which the distal tip 194 extends proximally to a cylindrical portion 198. The third component 180 may or may not be cannulated for use of a guide wire (not shown) via the tip opening 193.

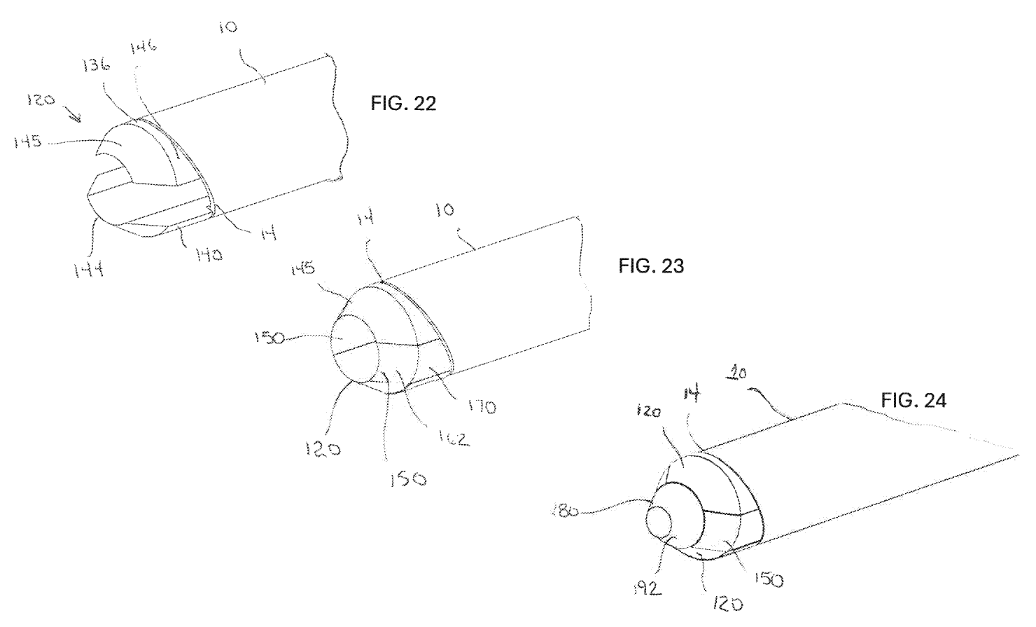

The interaction between the first, second and third components 120, 150, and 180 according to this embodiment is now further described with emphasis first being directed to the distal end of the described trocar apparatus 100 in FIGS. 22-24 and a cannula 10, FIG. 1.

First and with reference to FIG. 22, the first component 120 is axially advanced within the cannula 10 wherein the distal section 132 of the first component 120 is sufficiently flexible to permit passage through the hollow interior of the cannula 10 with minimal resistance. Once axially advanced beyond the tapered distal end 14 of the cannula 10, the distal section 132 of the first component 120, and more specifically the distal ends of the flexure-like members 136, 140, are then easily able to outwardly and radially expand, including the projecting blade portion 144, as shown. With reference to FIG. 13(b) and if the distal ends of the flexure-like members 145, 146, are manufactured as the flexed embodiment, the final radially expanded position will not be obtained until the non-compressible third component 180 is advanced through the distal end 14 of the cannula 10, thereby forcing radial expansion.

With reference to FIG. 23 and following the above movement of the first component 120, the distal end of the second component 150 is also sufficiently flexible enough such that the distal section 162 can pass through the hollow core or interior of the first component 120 with minimal resistance. Once beyond the distal end 14 of the outer cannula 10, the distal section 162 of the second component 150 is then easily able to outwardly and radially expand to a predetermined final position, the distal section 162 being oriented spatially with that of the distal section 132 of the first component 120 and the cannula 10, as shown, enabling the flexure-like members 166, 170 to also outwardly and radially expand. The flexure-like members 166, 170 are positioned orthogonally relative to the flexure-like members 136, 140, of the first component 120. As a result, the flexure-like members 166, 170 of the second component 150 "fit" within the removed annular wall portions of the first component 120, as shown. If the distal ends of the flexure-like members 176, are manufactured as the flexed embodiment as depicted in FIG. 17(b), the final radially expanded position will not be obtained until the non-compressible third component 180 is advanced beyond the distal end 14 of the cannula 10, thereby forcing radial expansion.

With reference to FIG. 24 and once axially advanced beyond the distal end 14 of the cannula 10, the distal section 192 of the third component 180 is axially pushed and rotated/twisted into a final position, which is oriented spatially with the distal sections 132, 162 of the first and second components 120, 150, respectively. The distal section 192 of the third component 180, which is incompressible may also provide an outward radial force to push the distal sections 132, 162, and more specifically the flexure-like members 136, 140 and 166, 170 of the first and second components 120, 150, respectively, into position, if these members are not already in the desired position. Additionally, the distal section 192 of the third component 180 can be configured to prevent an inward radial force from compressing the assembly 100 and displacing the first and second components 120, 150. In this position, the expanded trocar tip can engage the distal end 14 of the cannula 10, but without an abrupt shoulder or step being formed therebetween.

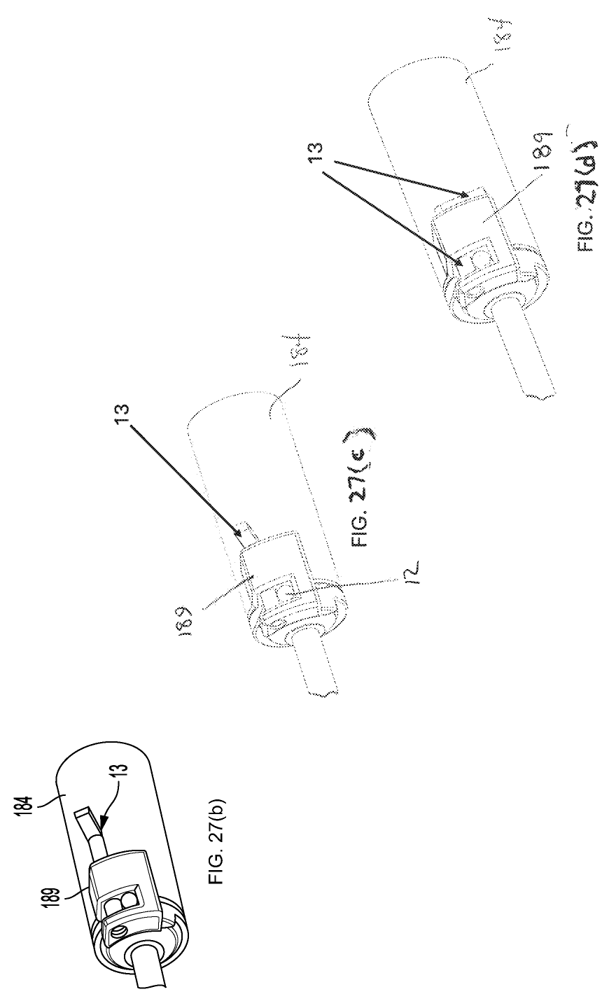

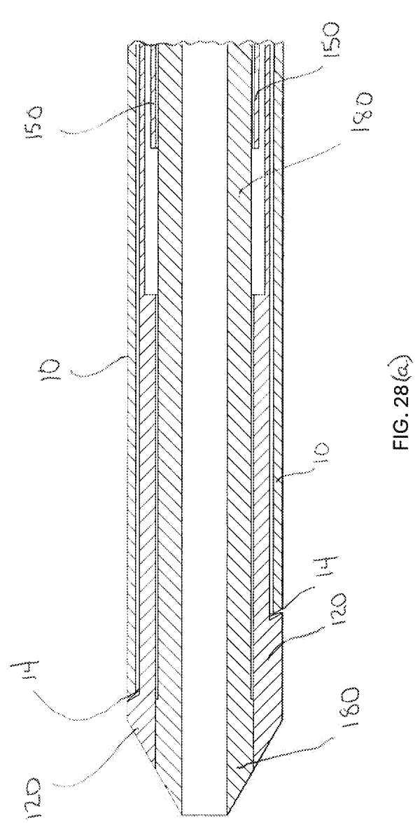

FIGS. 25-27(a) further illustrate the interaction between the first, second and third components 120, 150, 180 of the trocar assembly 100 in various stages of operation/use and more specifically the interaction of the proximal sections. In these herein described views, the cannula is not shown for ease of discussion. The above assembly 100, as described, provides a mechanism by which an expanded blade tip can be appropriately constructed and de-constructed. FIG. 25 depicts a starting or initial position (also referred to herein as the "expanded" position), while FIG. 26 depicts the trocar assembly 100 in a partially deployed (also referred to as a "partially compressed") position, and finally, FIG. 27(a) illustrates a fully deployed (also referred to as a "fully compressed") position of the herein described trocar assembly 100.

During axial compression of the assembly 100 which occurs after the first component 120 has engaged the cannula 10 fully, the second and third components 150, 180 move relative to the cannula 10 and the first component 120, which are fixed together. Rotational alignment of both components is maintained using protrusions 151 and 185 and slots 125 and 156, as previously indicated above. When fully compressed, protrusions 185 are then able to engage channel 157 of the second component 150 and rotate the third component 180 into its final position. This rotational movement allows the expansion 189 to engage the outward protrusion 12 on the cannula 10 through the hollowed portion 190. The structure of the expansion 189 is such that linear movement of the cannula/trocar assembly 10/100 are now all coordinated and the entire cannula/trocar assembly move as a single unit axially. However, as the insertion of the cannula/trocar assembly 100 into the body typically requires rotational, as well as linear movements, a locking mechanism to prevent rotation of the trocar assembly 100 relative to the cannula 10 is included. The expansion 189 on the proximal section 184 includes the slotted or hollowed region 190 in which the locking pin 13 can be accommodated. As is seen in FIG. 27(b), during assembly with the cannula 10, the locking pin 13 is located such that the expansion 189 can engage the outwardly extending protrusion 12 of the cannula 10. As seen in FIG. 27(c), once in position, the locking pin 13 is advanced to prevent de-rotation and disengagement until disassembly is required. FIG. 27(d) depicts the locking pin 13 having been rotated a quarter turn to lock it in position so that the locking pin 13 is suitably retained and does not easily fall out during use.

One embodiment of a suitable locking mechanism is discussed in greater detail below, although it will be readily apparent that there are other suitable mechanisms that can be used to provide temporary retention. As insertion of the cannula/trocar assembly typically requires a pushing and twisting motion by the user, the formed and expanded assembly tip resists rotational forces by being pushed against the body of the cannula 10 due to its slanted or tapered contour (30 degrees, for example) in addition to the locking mechanisms provided by the first and third components 120, 180. Many cannulas are contoured with a flat end and in these situations, the cannula tip would provide resistance to compressive, but not to rotational forces that can be placed on or applied to the trocar assembly 100. This compressive resistance would be provided by the elastic modulus of the material used and the locking mechanisms at the proximal sections of the various components.

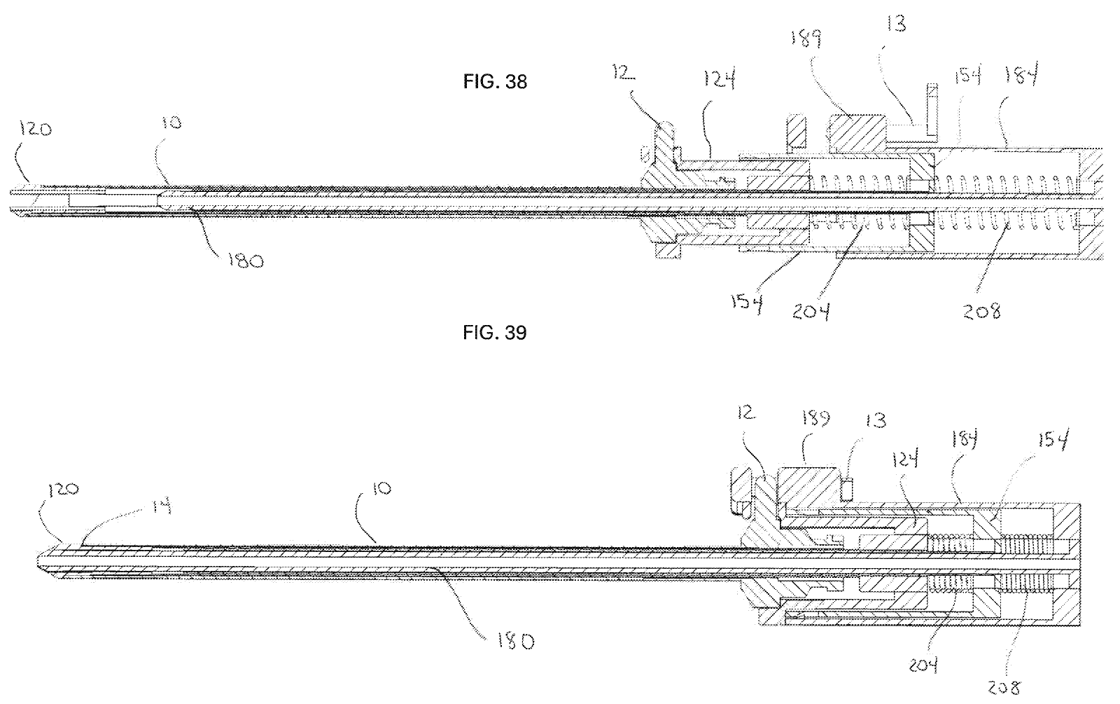

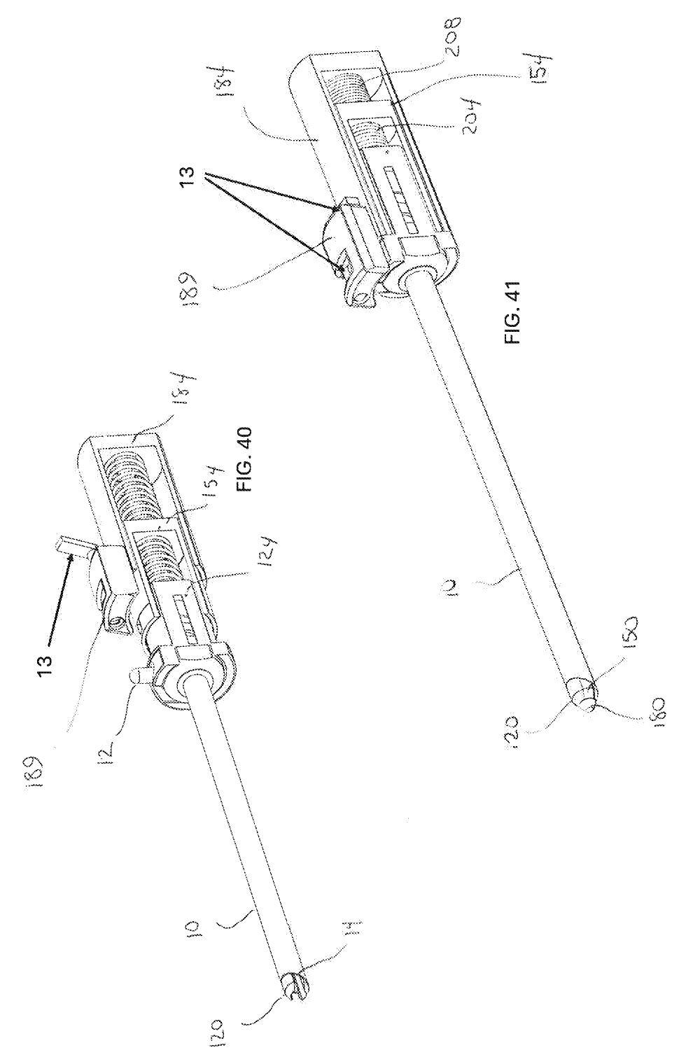

In order to achieve the appropriate result as also shown, by way of example, in FIG. 6, the order of movements of each of the first, second and third components 120, 150, 180 should be coordinated relative to the outer cannula 10. For instance and if the third component 180 were to advance too far linearly before the second component 150 was in its final position, then the trocar assembly 100 would jam within the interior of the cannula 10, as the outward radial force imparted by the third component 180 would force expansion within the confines of a normally rigid cannula. Therefore and to achieve a desired coordination between the various first, second and third components 120, 150, 180 of the herein described trocar assembly 100, a mechanism made up of springs having differential compressive moduli is provided, as more clearly shown in FIGS. 38-41. More specifically, a first compression spring 204 and a second compression spring 208 are provided. According to this embodiment, the first compression spring 204 is disposed between within the proximal section 154 of the second component 150 in which a distal end of the compression spring 204 engages the proximal end of the proximal portion 124 of the first component 120 and the proximal end of the spring 204 engages a proximal end of the proximal portion 154. The second compression spring 208 is linearly aligned with the first compression spring 204 and is disposed within the hollow interior of the proximal section 184 of the third component 180, in which a distal end of the spring 208 engages the outer proximal end of the second component 150, and a proximal end of the spring 208 engages the proximal end of the proximal portion 184 of the third component 180.

According to this embodiment and with reference to FIGS. 38-41, the first compression spring 204 is significantly more compressible than the second compression spring 208, such that the second component 150 can be advanced fully into its predetermined position with the distal end of the second component 150 advanced beyond the distal end 14 of the cannula 10 along with the already advanced first component 120 prior to substantial axial movement of the third component 180 relative to the second component 150 of the herein described assembly 100. It will be understood that other suitable mechanisms can be utilized for this purpose to provide this coordination, delay premature axial movement of the third component 180, and prevent potential jamming of the trocar assembly 100.

During insertion into the cannula prior to use, the trocar assembly 100 is in an uncompressed state and the first component 120 is easily deployed or expanded by insertion within the cannula 10 by axial movement and rotation to its final position. With further compression by axial movement of the second and third components 150, 180 toward the first component 120, the compression spring 204 housed between the first and second components 120, 150 being weaker and more easily compressed than the compression spring 208 between the second and third components 150, 180, allows the second component 150 to engage into its final predetermined position before the third component 180 can cause a jam within the cannula 10. The reverse occurs on disengagement of the various components to permit release and retraction within the cannula for removal of the trocar assembly 100. It will be understood that alternative mechanisms can be utilized for purposes of locking the various components and are not limited to this example.

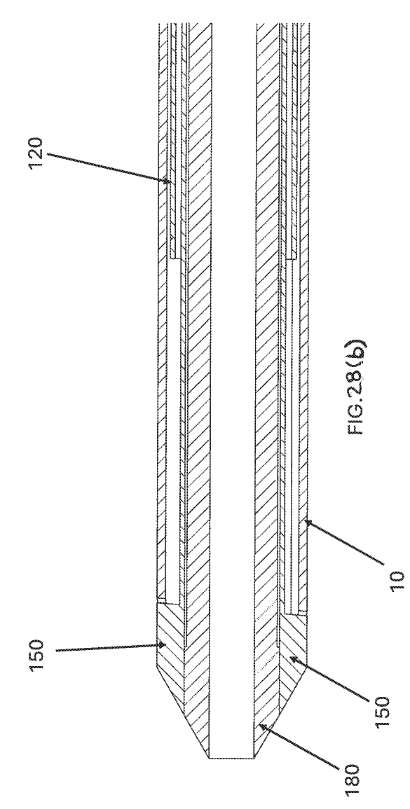

FIGS. 28(a) and 28(b) shows a longitudinal section through the distal end of the trocar/cannula assembly 100, taken in the vertical and horizontal planes, respectively, when the assembly is in the fully compressed position of FIG. 27(a), the assembly 100 being positioned for insertion into a bodily tissue. As is shown and when fully deployed, the tapered proximal edge is aligned with the tapered distal end 14 of the cannula 10 via the expanded trocar tip formed by the inter-connected first, second and third components 120, 150, 180.

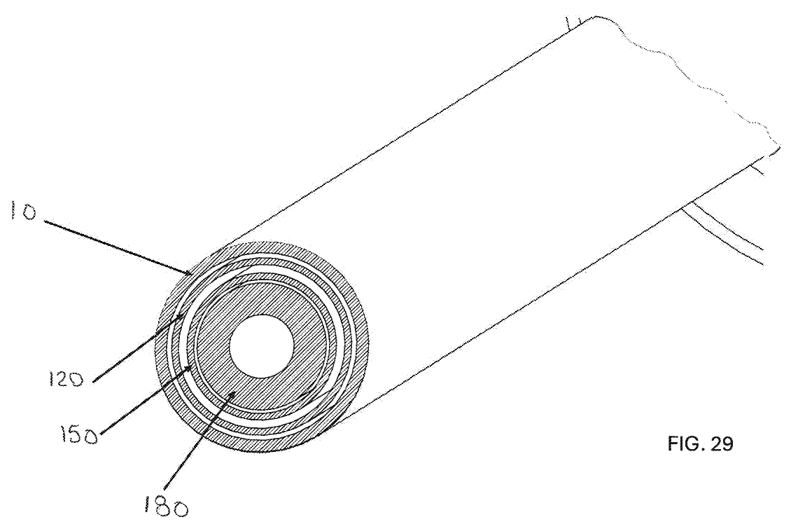

FIG. 29 shows a sectioned view taken through the various middle or intermediate sections of the trocar assembly 100, and more specifically depicting the nesting of the concentric shafts of each component 120, 150, 180 of the herein described assembly 100 according to this embodiment. Spacing between the components 120, 150, 180 is provided to prevent significant friction during insertion and removal thus ensuring smooth movements of all components.

Once locked in the desired position as shown in FIGS. 24 and 27(a), the trocar assembly 100 and outer cannula 10 can be used to penetrate the body tissues of a patient during a surgical procedure. Longitudinal force is resisted by the expanded tip being pushed against the body of the cannula 10, as well as the previously described locking mechanisms that are provided by the first, second and third components 120, 150 and 180 at the proximal sections of each, as previously described. Rotational force is prevented as well based on the interconnection between the first and third components 120, 180 and the cannula 10.

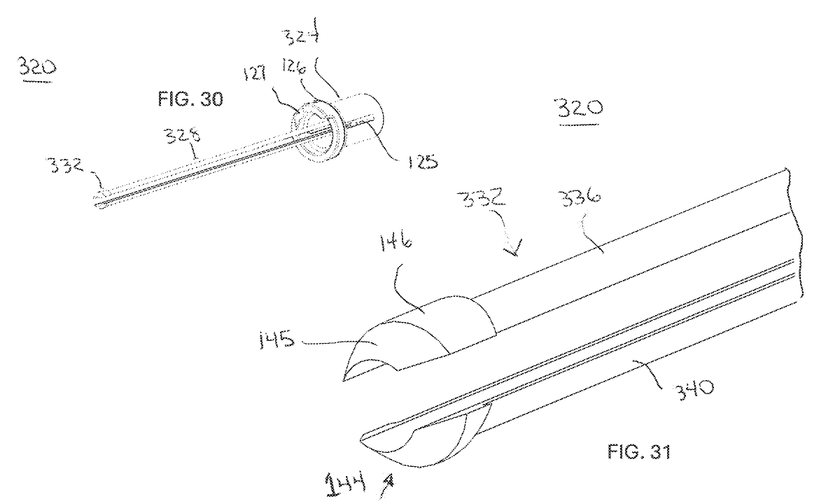

It should be noted that the concentric design of the middle or intermediate sections, as described above, is only one design or embodiment by which an expanded trocar tip can be created. For example and with small cannulas with inner diameters of around 5 mm or less, the ability to manufacture relatively long and thin tubes becomes a potential concern. Accordingly, FIGS. 30-37 depict aspects of an alternative design of a trocar assembly 300.

Similar to the first embodiment, the trocar assembly 300 is defined herein by a three (3) part assembly that is made up of respective interconnected and interlocked first, second and third components 320, 350, and 380. Each of the components are herein described in greater detail.

With reference to FIGS. 30 and 31, the first component 320 is formed of respective proximal, intermediate or middle, and distal sections 324, 328 and 332, respectively. The same reference numerals are herein used for the sake of clarity. More specifically, the proximal section 324 is similar to that described in the prior embodiment , The proximal section 324 of the first component 320 is configured so that the proximal section 324 can contour with the appropriate shape of a corresponding cannula, such as cannula 10, both distally and proximally. More specifically, the proximal section 324 is defined by a substantially cylindrical configuration that is essentially hollow. A raised distal end 126 a bayonet slot 127 disposed about the circumference of the raised distal end 126. The remainder of the proximal section 324 according to this specific embodiment is defined by a continuous cylindrical wall with the exception of a pair of axial slots 125 that are positioned in spaced relation diametrically and extending over substantially the length of the proximal section 324.

Each of the distal and intermediate sections 332, 328 of the first component 320 are defined primarily of a hollow tubular construction. However and unlike the prior embodiment, annular wall portions of the tubular construction are removed over substantially the entire length of each of these sections 328, 332, thereby creating a pair of flexure portions 336 and 340 that extend over the entire length of the intermediate and distal sections 328 and 332, respectively, as most clearly shown in FIG. 30. As in the prior described embodiment, the intermediate portion 328 bridges the distal portion 332 and the proximal portion 324, with the proximal end of the intermediate portion 328 extending into the hollow interior of the proximal section 324.

In addition to the flexure portions 336, 340, the distal most end of each of the flexure-like members 336, 340 is further defined by a blade portion 144, the latter portion 144 being made up of a frusto-conical distal tip 145 extending proximally to a raised cylindrical portion 146. According to this embodiment, the raised cylindrical portions 146 of each of the flexure-like members 336, 340 are defined by a tapered proximal end surface 148 wherein the overall length of the cylindrical portion 146 of one of flexure-like members 336 is axially longer than the overall length of the remaining flexure-like member 340. The tapered proximal end surface 148 creates an angled annular protrusion extending above the outer surface of the remaining proximal portion of the distal section 332 of the first component 320, which is defined by a cylindrical cross section.

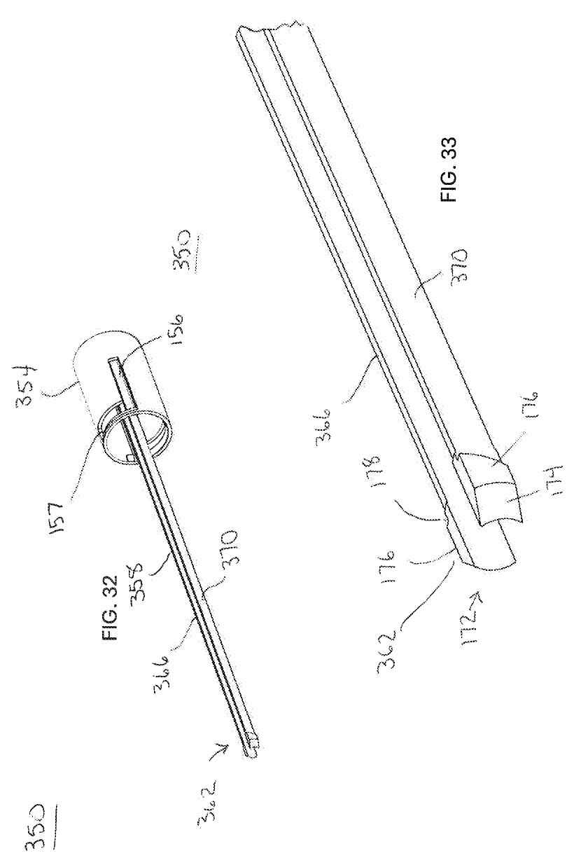

The second component 350 also includes respective proximal, middle and distal sections, 354, 358, 362, respectively, that are also similar to those in the previously described embodiment with reference to FIGS. 32 and 33. As previously noted, the same reference numbers are used for the sake of clarity to identify similar components or features.

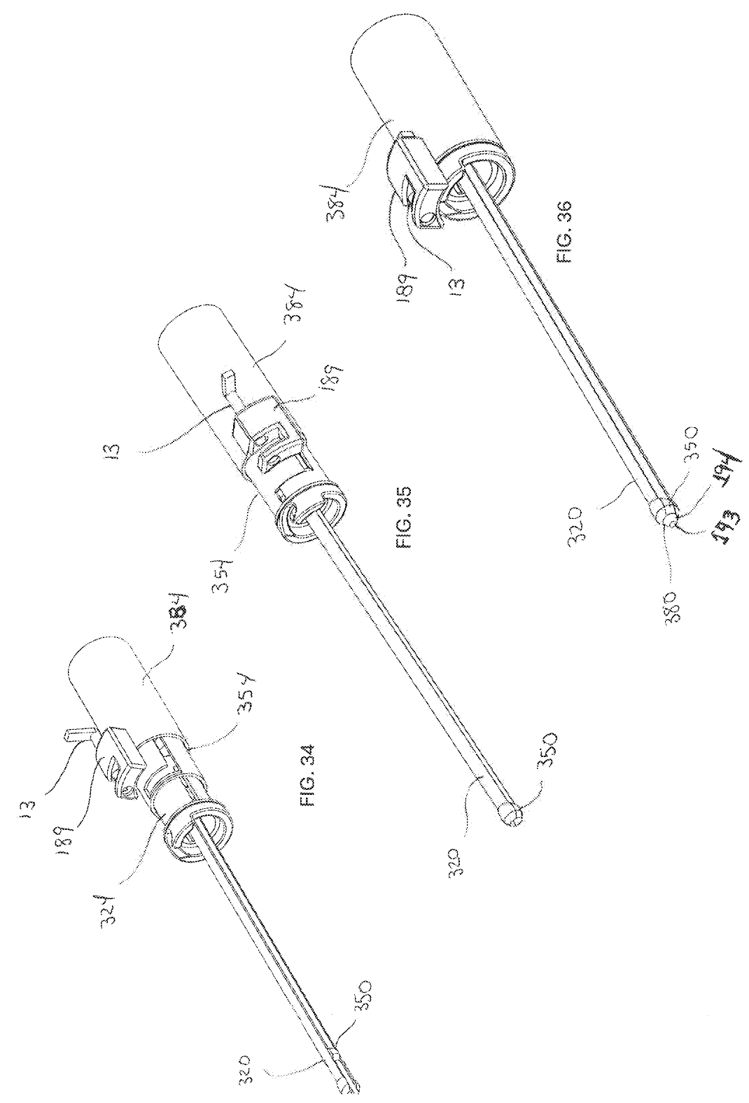

The proximal section 354 of the second component 350 is designed and appropriately sized to permit the proximal section 354 to slide over the outer surface of the proximal section 324 of the first component 320, as shown most clearly in FIG. 34. More specifically and according to this embodiment, the proximal section 354 is defined by a cylindrical hollow configuration. A pair of annular slots 157 are formed at the distal end of the proximal section 354, as well as a pair of linear slots 156 formed at one end of one of the annular slots 157 and forming an L-shaped configuration. According to this embodiment, the axial slots 156 extend proximally along the major axis of the proximal section 354.

The middle or intermediate section 358 of the second component 350 is defined by a substantially tubular cross section, which bridges the proximal and distal sections 354, 362. More specifically, the tubular intermediate section 358 proximally extends into the hollow cylindrical proximal section 354, the latter having a significantly greater diameter than the remainder of the second component 350. The intermediate portion 358 is sized and configured to allow passage through the hollow interior or core of the first component 320, and further to allow passage of the third component 380 within the second component 350, as described in greater detail below.

The distal section 362 of the second component 350 according to this embodiment is an extension of the cylindrical tubular cross section of the middle or intermediate sections 358 with the exception of removed annular wall portions, thereby defining a pair of flexure-like members 366, 370 in spaced and parallel relation to one another. Unlike the preceding embodiment, however, the flexure-like members 366, 370 extend over the entire length of the distal and intermediate sections 362, 354. Each of the flexure-like members 366, 370 are defined by a blade head 172 formed at the distal most end of the second component 350. The blade head 172 includes a beveled distal tip 174 at an open end of the second component 350 that extends to a raised annular portion 176. The raised annular portion 176 includes a proximal end surface 178 downwardly extending to the cylindrical outer surface of the second component 350. The flexure- like members 376, 380 of the second component 350 are sized to "fit" within the gaps or openings formed in the distal section 332 of the first component 320 when the second component 320 is axially advanced through the distal end of the cannula 10, thereby forming a fully cylindrical distal section of the assembly 300, when assembled and as described in greater detail below.

The third component 380, according to this embodiment, is unchanged in design from the that of the prior described trocar apparatus 100. This particular design of the first and second components 320, 350, among other differences, permit the cross-section of each of the components to have increased thickness, assuming that the components are fabricated from the same material, and thus potentially more easily manufacturable and durable than those previously described.

Operationally, the first component 320 is first inserted within the confines of a cannula 10. As the distal section 332 of the first component 320 advances beyond the distal end 14 of the cannula 10, the flexure-like members 336, 340 at the distal end of the first component 320 are caused to outwardly and radially expand. In one embodiment, where the members are manufactured in a flexed position, such as those previously shown in FIGS. 13(b) and 17(b), the outward and radial expansion does not occur until forced by the insertion of the third non- compressible component 380. The second component 350 is then advanced axially, but in this specific embodiment, the second component 350 is not concentrically positioned for deployment, but rather is arranged in a parallel configuration. The third component 380 is then sequentially advanced through the first and second components 320, 350, wherein the distal section 362 creates a radial outward force, ensuring the blade tip is properly expanded in relation to the cannula.

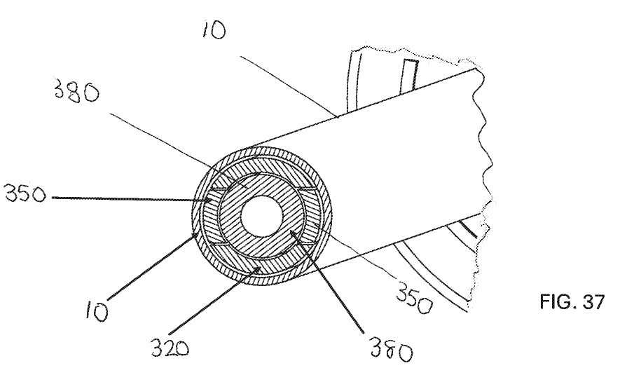

FIGS. 34-36 illustrate the assembly without a cannula for clarity with showing the sequential advancement of the first, second and third components 320, 350, 380, respectively. A sectioned view of the assembly is shown in FIG. 37 that is taken through the intermediate sections of the trocar assembly 300 and cannula 10.

The third component 380 is then advanced in a manner similar to that of the prior embodiment in which the distal portion of the third component 380 is rotated about its primary axis by movement of the proximal portion. The herein described components are locked in this predetermined position based on interlocking features provided, according to this specific embodiment, on the proximal portions of the first second and third components 320, 350, 380. In addition, the herein described assembly 300 can also include a mechanism similar to that previously described to promote coordination of the three components 320, 350, 380, as each are advanced through the hollow tubular cannula 10 in which axial movement of the third component 380 can be delayed to prevent jamming of the herein described trocar assembly 300, as shown in FIGS. 38-41, using a pair of compression springs 204, 208, each spring having a different compression modulus that enables coordinated movement of the assembly 300, in a manner as previously described.

Ultimately and in the predetermined and locked position, the formed blade tip of the trocar assembly 300 at the distalmost end of the first and second components 320, 350 is substantially smooth and does not create any substantial incongruence relative to the outer diameter of the cannula 10. Removal of the trocar assembly 300 from the cannula 10 is done in a reverse manner with the assembly 300 first being unlocked by rotational movement of the third component 380 by moving same from the formed annular slot 157 in the proximal section 354 of the second component 350. Movement is easily permitted as the preload on the compression springs 204, 208 is relieved according to this embodiment, and with the first, second and third components 320, 350, 380 being sequentially retracted axially from the cannula 10.

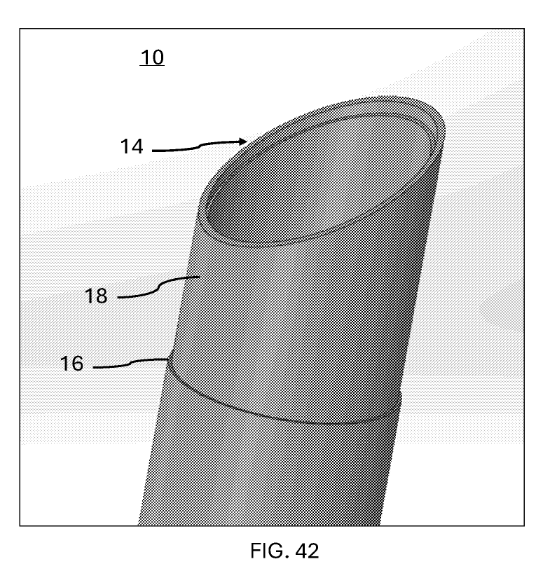

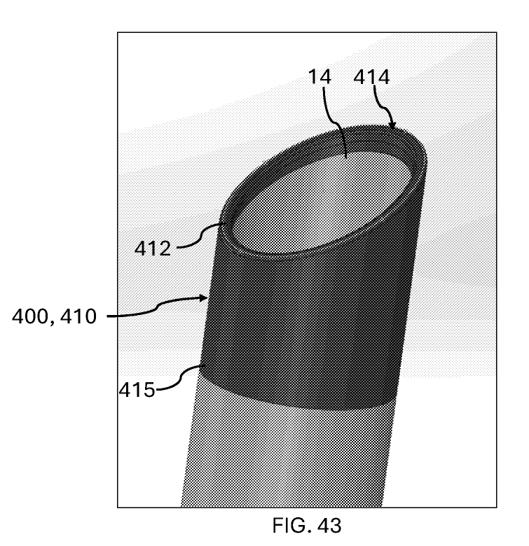

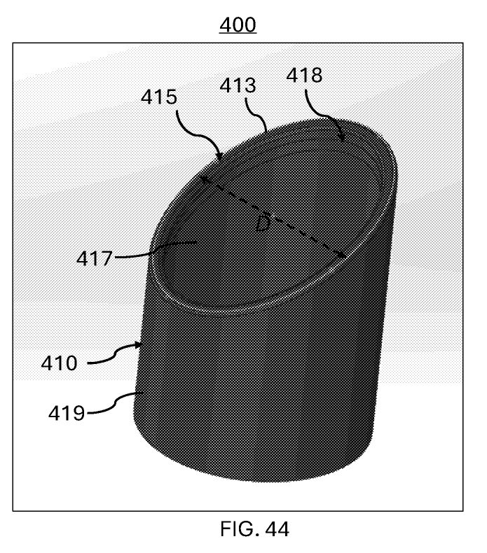

Referring to FIG. 42, an embodiment of a cannula 10 is shown including a distal tip end 14. A shoulder 16 is defined about the circumference of the body of the cannula 10 and is structured to form a seat region 18. Referring to the embodiments of FIGS. 43 and 44, the seat region 18 of the cannula 10 is structured to accept a sheath or sleeve 400 including a sheath body 410 in order to protect biological tissues during insertion and retraction of the cannula 10. In some embodiments, the sheath body 410 extends from a distal end 412 to a proximal end 415 such that the proximal end 415 abuts the shoulder 16 (FIG. 42). In some embodiments, the distal end 412 extends past the tip end 14 of the cannula 10 and includes a rim 413 that defines a sheath opening 415, which enables access into the interior of the cannula 10. In some embodiments, the distal end 412 of the sheath body 410 includes a tip end 414. In some embodiments, the shape of the tip end 414 corresponds to the shape of the tip end 14 of the cannula 10. In some embodiments, the rim 413 has a diameter D, that is at least the same diameter as the cannula opening at the tip end 14. In some embodiments, the sheath body has an interior surface 417 that is structured to define a bore 418 that traverses the body 410. In some embodiments, the interior surface is configured to contact the seat region 18 of the cannula 10 and an opposing exterior surface 419 of the body 410 faces away from the seat region 18 when the sheath 400 is installed onto the seat region 18 of a cannula 10.

In some embodiments, the sheath 400 is formed as a separate component from the cannula 10 and may be removed from the cannula 10. In some embodiments, the sheath 400 is coated onto or otherwise formed onto the seat region 18 of the cannula 10. In some embodiments the sheath cannot be removed from the cannula 10. In some embodiments, the cannula is formed from a biocompatible metal, such as stainless steel, and the sheath 400 is formed from a biocompatible plastic. In some embodiments, one or more components of the cannula 10, the first component 120, the second component 150, and the third component 180 may be at least partially coated with an anti-galling and/or a slippery coating. In some embodiments, the coating comprises one or more of the following: tungsten disulfide, molybdenum disulfide, polytetrafluoroethylene (PTFE), graphite, barium fluoride, graphene, hexagonal boron nitride.

While the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. Therefore, to the extent there are variations of the invention, which are within the spirit of the disclosure or equivalent to the inventions found in the claims, it is the intent that this patent will cover those variations as well.

To the extent that the claims recite the phrase "at least one of' in reference to a plurality of elements, this is intended to mean at least one or more of the listed elements, and is not limited to at least one of each element. For example, "at least one of an element A, element B, and element C," is intended to indicate element A alone, or element B alone, or element C alone, or any combination thereof. "At least one of element A, element B, and element C" is not intended to be limited to at least one of an element A, at least one of an element B, and at least one of an element C.

This Detailed Description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

The terminology used herein is for the purpose of describing particular embodiments only and is not intended to be limiting. As used herein, the singular forms "a," "an," and "the" are intended to include the plural forms as well, unless the context clearly indicates otherwise. It will be further understood that the terms "comprise" (and any form of comprise, such as "comprises" and "comprising"), "have" (and any form of have, such as "has" and "having"), "include" (and any form of include, such as "includes" and "including"), and "contain" (and any form of contain, such as "contains" and "containing") are open-ended linking verbs. As a result, a method or device that "comprises," "has," "includes," or "contains" one or more steps or elements possesses those one or more steps or elements, but is not limited to possessing only those one or more steps or elements. Likewise, a step of a method or an element of a device that "comprises," "has," "includes," or "contains" one or more features possesses those one or more features, but is not limited to possessing only those one or more features. Furthermore, a device or structure that is configured in a certain way is configured in at least that way, but may also be configured in ways that are not listed.

The corresponding structures, materials, acts, and equivalents of all means or step plus function elements in the claims below, if any, are intended to include any structure, material, or act for performing the function in combination with other claimed elements as specifically claimed. The description set forth herein has been presented for purposes of illustration and description, but is not intended to be exhaustive or limited to the form disclosed. Many modifications and variations will be apparent to those of ordinary skill in the art without departing from the scope and spirit of the disclosure. The embodiment was chosen and described in order to best explain the principles of one or more aspects set forth herein and the practical application, and to enable others of ordinary skill in the art to understand one or more aspects as described herein for various embodiments with various modifications as are suited to the particular use contemplated and in accordance with the following appended claims. Additional embodiments include any one of the embodiments described above and described in any and all exhibits and other materials submitted herewith, where one or more of its components, functionalities or structures is interchanged with, replaced by or augmented by one or more of the components, functionalities or structures of a different embodiment described above and as set forth in the following appended claims.

### PARTS LIST FOR FIGS. 1-41

10 cannula

12 protrusion, cannula

13 locking pin

14 distal end, cannula

15 handle portion, locking pin

16 shoulder, cannula

17 pin portion, locking pin

18 seat region, cannula

19 protrusion, locking pin

20 trocar

22 sharpened distal end, trocar

30 trocar

32 small hole

40 assembly

44 transition zone, step/shoulder

100 trocar assembly

120 first component

124 proximal section, first component

125 axial slots, proximal section

126 raised distal end, proximal section

127 bayonet slot

128 middle or intermediate section, first component

132 distal section, first component

136 flexure-like member

140 flexure-like member

144 blade portion

145 frusto-conical distal end

146 cylindrical portion

148 proximal end surface

150 second component

151 wedge shaped protrusions

154 proximal section, second component

156 axial slots, proximal section

157 annular slots, proximal section

158 middle or intermediate section, second component

162 distal section, second component

166 flexure-like member

170 flexure-like member

172 blade head

174 beveled distal end or tip

176 raised annular portion

178 proximal end surface

180 third component

184 proximal section, third component

185 wedge shaped protrusions

188 middle or intermediate section, third component

189 expansion

190 hollowed or slotted region

192 distal section, third component

193 opening, distal

194 frusto-conical distal tip

198 cylindrical portion

204 compression spring

208 compression spring

300 trocar assembly

320 first component

324 proximal section, first component

325 axial slot, proximal section

326 raised distal end, proximal section

327 bayonet slot

328 middle or intermediate section, first component

332 distal section, first component

336 flexure-like member

340 flexure-like member

350 second component

354 proximal section, second component

358 middle or intermediate section, second component

362 distal section, second component

366 flexure-like member

370 flexure-like member

380 third component

384 proximal section, third component

392 distal section, third component

400 sheath

410 body, sheath

412 distal end, body

413 rim, body

414 proximal end, body

415 opening, body

417 interior surface, body

419 exterior surface, body

It will be understood that there are a number of variations and modifications that be made in accordance with the herein described apparatus and related methods of use and as set forth in the following appended claims.

1. A trocar assembly sized and configured for penetrating body tissue of a patient without substantially damaging same, the trocar assembly comprising:
a hollow first component having a proximal section, an intermediate tubular section and a distal section;
a hollow second component having a proximal section, an intermediate tubular section, and a distal section, the second component being movable within the hollow first component;
a third component having a proximal section, an intermediate tubular section, and a distal section, the third component being sized to enable movement through the hollow second component and in which a hollow cannula is disposed over the first component such that the distal sections of the first second and third components are axially advanced thorough a distal opening of the cannula, the distal sections each being radially expanded such there is a smooth transition between the expanded trocar tip and the distal end of the cannula.

2. The trocar assembly according to claim 1, wherein the distal section of the first component is defined by flexure members that are radially expandable once advanced beyond the distal end of the cannula.

3. The trocar assembly according to claim 2, wherein the distal section of the first and second components are defined by flexure members that are radially expandable once advanced beyond the distal end of the cannula.

4. The trocar assembly according to claim 3, wherein flexure members of the second component advance between the flexure members of the first component when the second component is axially advance from the distal end of the cannula, thereby creating an expanded blade tip.

5. The trocar assembly according to claim 4, wherein the distal portion of the third component is sized and configured to create an outward radial force on the distal portions of the first and second components when the third component is axially advanced to a predetermined position beyond the distal end of the hollow cannula.

6. The trocar assembly according to claim 4, including a locking mechanism for fixedly positioning the expanded blade tip upon expansion of same.

7. The trocar assembly according to claim 1, further comprising a mechanism configured to prevent movement of the third component until the second component and first component have first been advanced axially through the distal end of the cannula.

8. The trocar assembly according to claim 7, wherein the mechanism configured to prevent movement of the third component comprises at least two compression springs, wherein a first compression spring has a spring constant that is greater than that of a second compression spring.

9. The trocar assembly according to claim 1, including means for fixing the position of the first, second and third components when fully deployed into the predetermined position.

10. A method for using a trocar assembly having interrelated components in which a second component is axially movable through a first component and a third component is axially movable through the second component, the method comprising:
axially advancing the first component beyond a distal end of a cannula surrounding the first component, wherein a distal section of the first component comprises a pair of flexure-like members;
axially advancing the second component of the trocar assembly within the first component and beyond the distal end of the cannula, wherein a distal section of the second component comprises a pair of flexure-like members aligned with the pair of flexure-like members of the first component;
axially advancing the third component within the second component and beyond the distal end of the cannula, wherein a distal section of the third component applies an outward radial force on the flexure-like members of the first and second components radially expanding a blade portion formed on the first and second component, such that when expanded the blade portion has an outer diameter that is consistent with the outer diameter of the cannula, thereby creating a substantially smooth transition zone.

11. The method according to claim 10, further comprising the step of restricting the movement of the third component until the second component has been fully advanced in relation to the first component within the cannula.

12. The method according to claim 11, wherein the restricting step comprises engagement of compression springs having different moduli in which a first compression spring has a spring constant that is greater than a spring constant of a second compression spring.

13. The method according to claim 10, further comprising the step of rotationally and axially fixing the first, second and third components in a predetermined position after the first second and third components have been first advanced beyond the distal end of the cannula.

14. The method according to claim 10, wherein the third component imparts a radial force ensuring the distal sections of the first and second component have been expanded to a predetermined position.

15. The method according to claim 10, including manufacturing the distal section of at least one of the first and second components in a flexed configuration.
